SEQUENCE LISTING (2 sequences)

SEQ ID NO: 1 - DNA, 20 bp, synthetic construct
  misc_feature 1..20: note = Synthetic
  source 1..20: mol_type = other DNA
gtaacatgtgccctttcctt

SEQ ID NO: 2 - DNA, 20 bp, synthetic construct
  misc_feature 1..20: note = Synthetic
  source 1..20: mol_type = other DNA
ccttccctgaaggttcctcc

(19) United States
(12) Patent Application Publication
(10) Pub. No.: US 2025/0099466 A1
(43) Pub. Date: Mar. 27, 2025

(54) MILVEXIAN FOR PREVENTION AND TREATMENT OF THROMBOEMBOLIC DISORDERS

(71) Applicants: BRISTOL-MYERS SQUIBB COMPANY, Princeton, NJ (US); JANSSSEN PHARMACEUTICA NV, Beerse (BE)

(72) Inventors: John STRONY, Raritan, NJ (US); Gary PETERS, Raritan, NJ (US); Madhu CHINTALA, Springhouse, PA (US); Christopher NESSEL, Bridgewater, NJ (US); Liyanage Vidya PERERA, Princeton, NJ (US); Danshi LI, Princeton, NJ (US); Joseph M. LUETTGEN, Princeton, NJ (US); Dietmar Alfred SEIFFERT, Princeton, NJ (US); Charlotte JONES-BURTON, Princeton, NJ (US)

(21) Appl. No.: 18/692,560

(22) PCT Filed: Sep. 16, 2022
(86) PCT No.: PCT/US2022/043797

§ 371 Date: Mar. 15, 2024

Related U.S. Application Data
(60) Provisional application No. 63/245,522, filed on Sep. 17, 2021.
(60) Provisional application No. 63/278,582, filed on Nov. 12, 2021.

(51) Int. Cl. A61K 31/506; A61P 7/02

(52) U.S. Cl. CPC A61K 31/506; A61P 7/02

(57) ABSTRACT

The factor Xia inhibitor milvexian has therapeutic properties useful in the treatment and/or prevention of thrombotic and thromboembolic disorders.

### CROSS-REFERENCE TO RELATED APPLICATIONS

This application is the United States National Phase of International Application No. PCT/US2022/043797, filed Sep. 16, 2022, which claims the benefit of U.S. Provisional Application No. 63/245,522, filed Sep. 17, 2021, and U.S. Provisional Application No. 63/278,582, filed Nov. 12, 2021. Each aforementioned application is incorporated by reference herein in its entirety.

### REFERENCE TO ELECTRONIC SEQUENCE LISTING

The application contains a Sequence Listing which has been submitted electronically in .XML format and is hereby incorporated by reference in its entirety. Said .XML copy, created on Oct. 13, 2022, is named “082867.000374.xml” and is 3,577 bytes in size. The sequence listing contained in this .XML file is part of the specification and is hereby incorporated by reference herein in its entirety.

### TECHNICAL FIELD

The disclosure pertains to the use of milvexian for the treatment of thromboembolic disorders.

### BACKGROUND

Oral anticoagulants are a mainstay for prevention and treatment of venous and arterial thromboembolism. Although direct oral anticoagulants have replaced vitamin K antagonists for many indications, bleeding remains the major side effect. Fear of bleeding contributes to the underuse of anticoagulants in eligible patients with atrial fibrillation, and the inappropriate use of low dose direct oral anticoagulant regimens. (Steinberg et al., International trends in clinical characteristics and oral anticoagulation treatment for patients with atrial fibrillation: Results from the GARFIELD-AF, ORBIT-AF I, and ORBIT-AF II registries. Am Heart J 2017; 194:132-40; Sanghai et al., Rates of potentially inappropriate dosing of direct-acting oral anticoagulants and associations with geriatric conditions among older patients with atrial fibrillation: The SAGE-AF study. J Am Heart Assoc 2020; 9:e014108). Therefore, a need for safer oral anticoagulants persists.

Factor XI is a promising target for development of new anticoagulants because it is an important driver of thrombus growth but plays a subsidiary part in hemostasis (Weitz et al., Factor XI inhibition to uncouple thrombosis from hemostasis: JACC review topic of the week. J Am Coll Cardiol 2021; 78:625-31).

A Phase 2 study for VTE prophylaxis post TKR provides the only published data on the clinical safety and efficacy of reducing FXI activity. Subjects were treated for 36 days prior to surgery with an FXI targeting antisense oligonucleotide (FXI-ASO, abelacimab) to reduce the level of FXI. The study showed a dose-dependent reduction in the risk of VTE events compared with enoxaparin. However, the antisense oligonucleotide and abelacimab require parenteral administration. (Buller et al. Factor XI antisense oligonucleotide for prevention of venous thrombosis. N Engl J Med 2015; 372:232-40; Verhamme et al. Abelacimab for prevention of venous thromboembolism. N Engl J Med 2021, 385(7): 609-617).

Milvexian is a direct-acting, reversible, small molecule therapeutic agent that binds to and inhibits the activated form of human coagulation Factor XI (FXIa) with high affinity and selectivity. Milvexian is a macrocyclic compound having the structure of Formula (I):

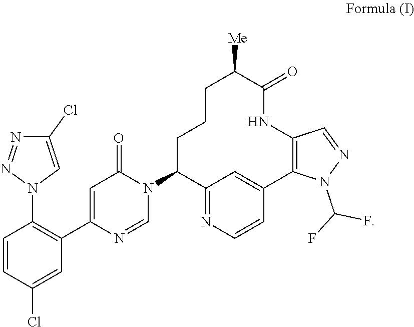 [SMILES: C[C@@H]1CCC[C@H](n2cnc(-c3cc(Cl)ccc3-n3cc(Cl)nn3)cc2=O)c2cc(ccn2)-c2c(cnn2C(F)F)NC1=O]

Milvexian has the chemical name (5R,9S)-9-(4-(5-chloro-2-(4-chloro-1H-1,2,3-triazol-1-yl)phenyl)-6-oxopyrimidin-1(6H)-yl)-21-(difluoromethyl)-5-methyl-21H-3-aza-1(4,2)-pyridina-2(5,4)-pyrazolacyclononaphan-4-one. Milvexian and a method of preparing milvexian are described in U.S. Pat. No. 9,453,018, which is hereby incorporated by reference in its entirety. Amorphous solid dispersion composition of milvexian in one or more polymers has been described in WO2020210629, which is hereby incorporated by reference in its entirety.

There is a need in the art for a new anticoagulant therapy that significantly reduces thrombosis, without increasing the risk of clinically significant bleeding, in patients that are either suffering from or at risk for thrombotic events.

### SUMMARY

In some aspects, the disclosure is directed to methods of treating or preventing a venous thromboembolic disorder, wherein the methods comprise orally administering to a patient in need thereof a total daily dose of from 25 mg to 400 mg of milvexian, or pharmaceutically acceptable salt thereof.

In other aspects, the disclosure is directed to methods of preventing post-operative venous thromboembolism events in a patient recovering from surgery, said method comprising administering to said patient a daily dose of milvexian, or pharmaceutically acceptable salt thereof, wherein said administration results in the patient experiencing fewer post-operative venous thromboembolism events than would result from subcutaneous administration to the patient of 40 mg per day of enoxaparin (a low molecular weight heparin).

### BRIEF DESCRIPTION OF THE DRAWINGS

FIG. 1 gives an overview of the study design of the study described in Example 1 which was designed to investigate the ability of milvexian to prevent venous thromboembolic events in individuals at risk of developing venous thromboembolism, e.g., individuals undergoing total knee arthroplasty. Interim analysis (IA) #1 was conducted when ˜50 subjects in each of the BID treatment groups had completed venography or had a symptomatic venous thromboembolism (VTE) event. IA #2 was an additional IA conducted at the discretion of the operations committee to determine the need for continuing the 25 mg, once a day, dose regimen.

FIG. 2 illustrates the total VTE incidence rate and 95% confidence interval for each treatment arm of the study described in Example 1.

FIG. 3 illustrates, of the subjects with deep vein thrombosis (DVT) on venography in the trial, incidence rate of clots by clot severity.

FIG. 4 illustrates, of the subjects with DVT on venography in the study described in Example 1, average DVT severity score.

FIG. 5 illustrates the incidence rate and 95% confidence interval for bleeding for each treatment arm of the study described in Example 1.

FIG. 6 illustrates the median activated partial-thromboplastin time ratios and rates of any bleeding or clinically relevant bleeding with milvexian and enoxaparin.

FIG. 7 illustrates effects of vehicle and milvexian given IV on carotid blood flow after thrombus induction in ECAT rabbits. Means±SEM and n=6 per group.

FIG. 8 illustrates effects of vehicle and milvexian on integrated blood flow expressed as % control carotid blood flow (i.e., pre-injury blood flow) in ECAT rabbits. *P<0.05 (one-sided) compared to vehicle. Means±SEM and n=6 per group.

FIG. 9 illustrates the relationship between total plasma concentrations of milvexian and antithrombotic effects expressed as % reduction of thrombus weight in prevention ECAT rabbits. IV, intravenous; ECAT, electrically mediated carotid arterial thrombosis; SEM, standard error of the mean.

FIG. 10 illustrates effects of vehicle and milvexian on aPTT, TT, and PT in ECAT rabbits. *P<0.05 (one-sided) compared to vehicle. Means±SEM and n=6 per group.

FIG. 11 illustrates tracking antithrombotic activity of milvexian with ex vivo aPTT in rabbit ECAT. aPTT, activated partial thromboplastin time; TT, thrombin time; PT, prothrombin time; ECAT, electrically mediated carotid arterial thrombosis; SEM, standard error of the mean; IV, intravenous.

FIG. 12 illustrates effects of vehicle and milvexian on carotid blood flow (expressed as % of control carotid blood flow). In the treatment protocol, vehicle or milvexian was given IV (bolus+infusion) at 15 minutes after the initiation of arterial thrombosis. Means±SE and n=6 per group.

FIG. 13 illustrates the dose-dependent decrease in FXI levels and clot weight observed in the rabbit ECAT model with ASO-induced inhibition of FXI.

### DETAILED DESCRIPTION OF ILLUSTRATIVE EMBODIMENTS

As used herein, the term “treating” or “treatment” includes the therapeutic treatment of thromboembolic disorders. The term “treating” or “treatment” covers the treatment of a disease-state in a mammal, particularly in a human, and include: (a) inhibiting the disease-state, i.e., arresting it development; and/or (b) relieving the disease-state, i.e., causing regression of the disease state. In some embodiments, milvexian is used for the postoperative treatment of VTE in patients undergoing surgery. In some embodiments, milvexian is used for the short term treatment of VTE (i.e., acute treatment of VTE).

As used herein, the term “prevention” or “preventing” refers to the preventive treatment of a subclinical disease-state in a mammal, particularly in a human, aimed at reducing the probability of the occurrence of a clinical disease-state. Patients are selected for preventative therapy based on factors that are known to increase risk of suffering a clinical disease state compared to the general population. In some embodiments, “prevention” covers primary prevention of thromboembolic disorders in a mammal, particularly in a human, aimed at reducing the probability of the occurrence of total VTE events after TKR surgery including: proximal and/or distal deep vein thrombosis (both symptomatic DVT and asymptomatic DVT confirmed by venography assessment or objectively confirmed symptomatic); nonfatal PE; or any death during the treatment period. Patients are selected for preventative therapy based on factors that are known to increase risk of suffering a clinical disease state compared to the general population. In some embodiments, milvexian is used for the preoperative thromboprophylaxis of VTE in patients undergoing surgery. In some embodiments, milvexian is used for the postoperative thromboprophylaxis of VTE in patients undergoing surgery.

As used herein, the term “therapeutically effective amount” is intended to include an amount of milvexian, or therapeutically effective salt, or solvate thereof, that is effective when administered alone to inhibit factor XIa and/or plasma kallikrein and/or to prevent or treat the disorders listed herein. In an embodiment, therapeutically effective amount is intended to include an amount of milvexian, or therapeutically effective salt thereof, that is effective when administered alone to inhibit factor XIa and/or plasma kallikrein and/or to prevent or treat the disorders listed herein. In an embodiment, therapeutically effective amount of milvexian is clinically proven effective in reducing the occurrence of total VTE events in a patient during treatment period. In an embodiment, therapeutically effective amount of milvexian comprises therapeutically effective solvate of milvexian, for example, the acetone monosolvate.

As used herein, the term “thrombosis”, refers to formation or presence of a thrombus (pl. thrombi); clotting within a blood vessel that may cause ischemia or infarction of tissues supplied by the vessel. The term “embolism”, as used herein, refers to sudden blocking of an artery by a clot or foreign material that has been brought to its site of lodgment by the blood current. The term “thromboembolism”, as used herein, refers to obstruction of a blood vessel with thrombotic material carried by the blood stream from the site of origin to plug another vessel. The term “thromboembolic disorders” entails both “thrombotic” and “embolic” disorders.

The term “thromboembolic disorders” as used herein also includes specific disorders selected from, but not limited to, atherosclerosis, atherothrombosis, peripheral occlusive arterial disease, venous thrombosis, venous thromboembolism (VTE), deep vein thrombosis, thrombophlebitis, coronary arterial thrombosis, kidney embolism, pulmonary embolism, and thrombosis resulting from medical implants, devices, or procedures in which blood is exposed to an artificial surface that promotes thrombosis.

In some embodiments, “thromboembolic disorders” includes specific disorders selected from, but not limited to, atherosclerosis, peripheral occlusive arterial disease, venous thrombosis, venous thromboembolism (VTE), deep vein thrombosis, thrombophlebitis, coronary arterial thrombosis, kidney embolism, pulmonary embolism, and thrombosis resulting from medical implants, devices, or procedures in which blood is exposed to an artificial surface that promotes thrombosis.

In another embodiment, the term “thromboembolic disorders” includes venous thromboembolism, deep vein thrombosis (DVT), proximal and/or distal DVT, symptomatic proximal and/or distal DVT, asymptomatic proximal and/or distal DVT, or pulmonary embolism.

In some embodiments, milvexian is used for the primary prophylaxis of VTE in patients undergoing surgery. In some embodiments, milvexian is used for the preoperative thromboprophylaxis of VTE in patients undergoing surgery.

In some aspects, the disclosure is directed to methods of treating or preventing a venous thromboembolic disorder, wherein the methods comprise orally administering to a patient in need thereof a clinically effective amount of milvexian, or pharmaceutically acceptable salt thereof. In some embodiments, the clinically effective amount of milvexian, or pharmaceutically acceptable salt thereof, may be a total daily dose of from 25 mg to 400 mg. In other embodiments, the clinically effective amount may be a total daily dose of from 50 mg to 400 mg.

In some aspects, the disclosure is directed to methods of treating or preventing a venous thromboembolic disorder, wherein the methods comprise orally administering to a patient in need thereof a total daily dose of from 25 mg to 400 mg of milvexian, or pharmaceutically acceptable salt thereof. In other embodiments, the total daily dose is from 50 mg to 400 mg of milvexian, or pharmaceutically acceptable salt thereof.

In some aspects, the methods of the disclosure are directed to treating or preventing a venous thromboembolic disorder. In some embodiments, the venous thromboembolic disorder is a composite of asymptomatic deep-vein thrombosis, confirmed symptomatic venous thromboembolism (symptomatic deep-vein thrombosis of the leg or nonfatal pulmonary embolism), or death.

In other embodiments, the venous thromboembolic disorder is proximal deep-vein thrombosis (symptomatic or asymptomatic), distal deep-vein thrombosis (symptomatic or asymptomatic), nonfatal pulmonary embolism, or death.

In other embodiments, the venous thromboembolic disorder is proximal and/or distal deep vein thrombosis, nonfatal pulmonary embolism, or death.

In other embodiments, the venous thromboembolic disorder is proximal and/or distal deep vein thrombosis.

In other embodiments, the venous thromboembolic disorder is nonfatal pulmonary embolism.

In other embodiments, the venous thromboembolic disorder is death.

In some aspects, the disclosure is directed to methods of preventing post-operative venous thromboembolism events in a patient recovering from surgery, wherein the method comprises administering to the patient a daily dose of milvexian, or pharmaceutically acceptable salt thereof, wherein the administration results in the patient experiencing fewer post-operative venous thromboembolism events than would result from subcutaneous administration to the patient of 40 mg per day of enoxaparin.

In some embodiments of such methods, the patient is at risk of experiencing post-operative venous thromboembolism events such as the venous thromboembolic disorders described above. In some embodiments, the patient is recovering from a surgical procedure. In some embodiments, the patient is recovering from abdominal surgery, knee replacement surgery, or hip replacement surgery.

In such methods, the patient may be administered milvexian, or pharmaceutically acceptable salt thereof, beginning either just prior to undergoing the surgical procedure, or beginning just after the surgical procedure.

In some embodiments of these methods, the patient experiences fewer post-operative venous thromboembolism events than would result from subcutaneous administration to the patient of 40 mg per day of enoxaparin. That is, the risk of the patient experiencing a post-operative venous thromboembolism event is lower than it would have been had the patient been administered, instead of milvexian or pharmaceutically acceptable salt thereof, 40 mg of enoxaparin administered subcutaneously.

In some embodiments of these methods, the risk ratio is 0.6 or less, such as, for example, 0.55 or less, 0.5 or less, 0.45 or less, 0.4 or less, 0.35 or less, or 0.3 or less. That is, in such methods, the risk of the patient experiencing a post-operative venous thromboembolism event is 0.6 times or less the risk the patient would have experienced had the patient been administered 40 mg enoxaparin subcutaneously.

In some aspects, the disclosure is directed to methods of treating or preventing thrombosis resulting from medical implants, devices, or procedures in which blood is exposed to an artificial surface that promotes thrombosis, wherein the methods comprise administering to the patient a daily dose of a therapeutically effective amount of milvexian, or pharmaceutically acceptable salt thereof.

In some aspects, the disclosure provides methods of treating or preventing a venous thromboembolic disorder comprising: orally administering therapeutically effective amount of milvexian, or pharmaceutically acceptable salt thereof once or twice daily to a patient in need thereof, wherein an incidence rate of total venous thromboembolism events in the patient treated with the milvexian, or pharmaceutically acceptable salt thereof is less than 25% with an alpha of 5%.

In some embodiments, for any of the methods described herein, the treatment with milvexian in a patient in need thereof causes a statistically significant reduction in total VTE events (p<0.0001 (one-sided)) as compared with the incidence rate of total VTE with enoxaparin.

In some embodiments, for any of the methods described herein, the treatment with milvexian in a patient in need thereof prevents the occurrence of the total VTE events in dose-dependent response manner and the occurrence of the total VTE events is at an rate statistically lower than 30% with an alpha of 5%; wherein the total VTE events include proximal and/or distal DVT (asymptomatic confirmed by venography assessment or objectively confirmed symptomatic), nonfatal PE, or any death) during the treatment period.

In other embodiments, for any of the method described herein, the occurrence of the total VTE events in the patient is at a rate statistically lower than 25% with an alpha of 5%.

In other embodiments, for any of the method described herein, the occurrence of the total VTE events in the patient is at a rate statistically lower than 20% with an alpha of 5%.

In other embodiments, for any of the method described herein, the occurrence of the total VTE events in the patient is at a rate statistically lower than 15% with an alpha of 5%.

In other embodiments, for any of the method described herein, the occurrence of the total VTE events in the patient is at a rate statistically lower than 10% with an alpha of 5%.

In some aspects, the disclosure provides methods of treating or preventing a venous thromboembolic disorder comprising: orally administering therapeutically effective amount of milvexian, or pharmaceutically acceptable salt thereof once or twice daily to a patient in need thereof, wherein an incidence rate of total venous thromboembolism events in the patient treated with the milvexian, or pharmaceutically acceptable salt thereof is less than 25% by the modified intention-to-treat population with a one-sided alpha of 5% and CI=95%.

In some embodiments, for any of the methods described herein, the treatment with milvexian in a patient in need thereof causes a statistically significant reduction in total VTE events (p<0.0001 (one-sided)) as compared with the incidence rate of total VTE with enoxaparin.

In some embodiments, for any of the methods described herein, the treatment with milvexian in a patient in need thereof prevents the occurrence of the total VTE events in dose-dependent response manner and the occurrence of the total VTE events is at an rate statistically lower than 30% by the modified intention-to-treat population with a one-sided alpha of 5% and CI=95% (CI stands for Confidence Interval of the incidence rate); wherein the total VTE events include proximal and/or distal DVT (asymptomatic confirmed by venography assessment or objectively confirmed symptomatic), nonfatal PE, or any death) during the treatment period.

In other embodiments, for any of the method described herein, the occurrence of the total VTE events in the patient is at a rate statistically lower than 25% by the modified intention-to-treat population with a one-sided alpha of 5% and CI=95%.

In other embodiments, for any of the method described herein, the occurrence of the total VTE events in the patient is at a rate statistically lower than 20% by the modified intention-to-treat population with a one-sided alpha of 5% and CI=95%.

In other embodiments, for any of the method described herein, the occurrence of the total VTE events in the patient is at a rate statistically lower than 15% by the modified intention-to-treat population with a one-sided alpha of 5% and CI=95%.

In other embodiments, for any of the method described herein, the occurrence of the total VTE events in the patient is at a rate statistically lower than 10% by the modified intention-to-treat population with a one-sided alpha of 5% and CI=95%.

As used herein, the term “intention-to-treat population” (ITF population) refers to all randomized subjects who have signed an informed consent in a clinical trial such as that described in Example 1 below. As used herein, the term “modified intention-to-treat population” (mITT-CEC adjudicated population) refers to a subset of the ITT population consisting of subjects with a valid assessment of potential efficacy outcome and who take at least 1 dose of the study drug milvexian in a clinical study such as that described in Example 1 below.

In some embodiments of the methods disclosed herein, the therapeutically effective amount of milvexian, or pharmaceutically acceptable salt thereof is an amount that is clinically proven effective in achieving less than 25% of the incidence rate of total venous thromboembolism events in the patient treated with milvexian, or pharmaceutically acceptable salt thereof.

In some embodiments, the 50 mg to 400 mg amount of milvexian, or the pharmaceutically acceptable salt thereof was clinically proven effective in causing a statistically significant reduction in total VTE events (p<0.0001 (one-sided)) as compared with the incidence rate of total VTE events with enoxaparin.

In some aspects of the disclosed methods, milvexian, or pharmaceutically acceptable salt thereof, is orally administered to a patient.

The dose of milvexian, or pharmaceutically acceptable salt thereof, may be administered in any suitable oral dosage form, formulation or pharmaceutical formulation, such as, for example, tablets, capsules (including those that contain sustained release or timed release formulations), pills, powders, granules, elixirs, tinctures, suspensions, syrups, and emulsions.

In some embodiments, the dose of milvexian, or pharmaceutically acceptable salt thereof, administered may be formulated as immediate release formulation. In some embodiments, the dose of milvexian administered may be formulated as immediate release capsule formulation. In some embodiments, the dose of milvexian administered may be formulated as an amorphous solid dispersion composition of milvexian in one or more polymers. In some embodiments, the polymer in the amorphous solid dispersion is hypromellose acetate succinate (HPMCAS). In some embodiments, the amorphous solid dispersion composition of milvexian in one or more polymers is prepared by spray drying. In some embodiments, the amorphous solid dispersion composition of milvexian in one or more polymers comprises milvexian being molecularly dispersed in the one or more polymers.

In some aspects of the disclosed methods, the patient to whom milvexian, or pharmaceutically acceptable salt thereof, is administered is a mammal. In some embodiments, the patient is a human. In other embodiments, the patient is a male human. In other embodiments, the patient is a female human. In some embodiments, the patient is a human who is 50 years of age or older. In other embodiments, the patient is a human who is less than 50 years of age. In some embodiments, the patient is a human who is less than 18 years or age.

In some aspects of the disclosed methods, the patient is administered milvexian, or pharmaceutically acceptable salt thereof.

In some embodiments, the patient is administered milvexian.

In other embodiments, the patient is administered a pharmaceutically acceptable salt of milvexian.

As used herein, “pharmaceutically acceptable salt” refers to derivatives wherein a compound is modified by making an acid or a basic salts thereof. Examples of pharmaceutically acceptable salts include, but are not limited to, mineral or organic acid salts of basic groups such as amines; and alkali or organic salts of acidic groups such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic. The pharmaceutically acceptable salts of milvexian can be synthesized using conventional chemical methods. Generally, such salts can be prepared by reacting t milvexian with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 18th Edition, Mack Publishing Company, Easton, Pa. (1990), the disclosure of which is hereby incorporated by reference.

In embodiments of the disclosed methods in which a pharmaceutically acceptable salt of milvexian is administered, then the amount specified is on a milvexian basis. That is, an amount of the pharmaceutically acceptable salt is administered that contains the specified amount of milvexian. For example, administering 50 mg of milvexian, or a pharmaceutically acceptable salt thereof, refers to administering either 50 mg of milvexian, or an amount of a pharmaceutically acceptable salt of milvexian that contains 50 mg of milvexian.

In some aspects of the disclosed methods, the patient is administered a total daily dose of from 50 mg to 400 mg of milvexian, or pharmaceutically acceptable salt thereof.

As used herein, the term “total daily dose” refers to the total amount of milvexian administered in a day. Thus, the total daily dose represents the cumulative amount administered in all dosing episodes in a given day. For example, if a patient is administered 25 mg of milvexian at each of two dosing episodes on a given day, then the total daily dose for that day is 50 mg.

In some embodiments, for any of the method described herein, the milvexian, or pharmaceutically acceptable salt thereof, is administered wherein the total daily dose is administered in a single dose.

In some embodiments the single daily dose may be administered to the patient approximately once every 18-30 hours, once every 20-28 hours, once every 22-26 hours or once every 23 to 25 hours.

In other embodiments the single daily dose may be administered to the patient approximately once every 20 hours, once every 21 hours, once every 22 hours, once every 23 hours, once every 24 hours, once every 25 hours, once every 26 hours, once every 27 hours or once every 28 hours.

In other embodiments, for any of the method described herein, the milvexian, or pharmaceutically acceptable salt thereof, is administered wherein the total daily dose is administered in divided doses.

In other embodiments, for any of the method described herein, the milvexian, or pharmaceutically acceptable salt thereof, is administered wherein the total daily dose is administered in two doses.

In some embodiments in which the total daily dose is administered in two doses, the doses may be administered to the patient approximately once every 8-16 hours, once every 9-15 hours, once every 10-14 hours or once every 11-13 hours.

In some embodiments in which the total daily dose is administered in two doses, the doses may be administered approximately once every 10 hours, once every 10.5 hours, once every 11 hours, once every 11.5 hours, once every 12 hours, once every 12.5 hours, once every 13 hours, once every 13.5 hour or once every 14 hours.

In other embodiments, for any of the method described herein, the milvexian, or pharmaceutically acceptable salt thereof, is administered wherein the total daily dose is administered in more than two doses.

In some embodiments, for any of the methods described herein, milvexian, or the pharmaceutically acceptable salt thereof, may be administered to the patient once or twice daily for at least 10 consecutive days.

In other embodiments of any of the methods described herein, milvexian, or the pharmaceutically acceptable salt thereof, may be administered to the patient once or twice daily every day for at least 11 days, at least 12 days, at least 13 days, at least 14 days, at least 15 days, at least 30 days, at least 6 months, at least 1 year, or at least 5 years.

In embodiments in which milvexian, or a pharmaceutically acceptable salt thereof, is administered to prevent total venous thromboembolic events (VTE) in a patient undergoing knee or hip replacement surgery, it may be administered to the patient once or twice daily for at least 10 consecutive days, at least 11 consecutive days, at least 12 consecutive days, at least 13 consecutive days or 14 consecutive days.

In some embodiments, for any of the method described herein, the milvexian, or pharmaceutically acceptable salt thereof, is administered at a total daily dose ranging from 25 mg to 400 mg, such as, for example, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, or 400 mg.

In some embodiments, for any of the method described herein, the milvexian, or pharmaceutically acceptable salt thereof, is administered at a total daily dose ranging from 50 mg to 400 mg, such as, for example, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, or 400 mg.

In some embodiments, for any of the method described herein, the milvexian, or pharmaceutically acceptable salt thereof is administered at the total daily dose selected from the group consisting of 50 mg, 100 mg, 200 mg, and 400 mg.

In some embodiments, for any of the method described herein, the milvexian, or pharmaceutically acceptable salt thereof is administered at the total daily dose of 25 mg.

In some embodiments, for any of the method described herein, the milvexian, or pharmaceutically acceptable salt thereof is administered at the total daily dose of 50 mg.

In some embodiments, for any of the method described herein, the milvexian, or pharmaceutically acceptable salt thereof is administered at the total daily dose of at least 50 mg.

In some embodiments, for any of the method described herein, the milvexian, or pharmaceutically acceptable salt thereof is administered at the total daily dose of 100 mg.

In some embodiments, for any of the method described herein, the milvexian, or pharmaceutically acceptable salt thereof is administered at the total daily dose of at least 100 mg.

In some embodiments, for any of the method described herein, the milvexian, or pharmaceutically acceptable salt thereof is administered at the total daily dose of 200 mg.

In some embodiments, for any of the method described herein, the milvexian, or pharmaceutically acceptable salt thereof is administered at the total daily dose of at least 200 mg.

In some embodiments, for any of the method described herein, the milvexian, or pharmaceutically acceptable salt thereof is administered at the total daily dose of 400 mg.

In some embodiments, for any of the method described herein, the milvexian, or pharmaceutically acceptable salt thereof is administered at the total daily dose of at least 400 mg.

In some aspects of the methods described herein, there is no dose-dependent response of milvexian for the occurrence of the endpoint of Any Bleeding events during the 10-14 day treatment period.

In some embodiments of any of the methods described herein, administering milvexian, or the pharmaceutically acceptable salt thereof, to the patient does not increase the patient's incidence of bleeding, e.g., increase the patient's bleeding incidence by more than 1%, more than 2%, more than 3%, more than 4%, more than 5%, more than 6%, more than 7% or more than 8%.

In some embodiments of any of the methods described herein for treating or preventing a thromboembolic disorder, the administering of milvexian, or the pharmaceutically acceptable salt thereof, to the patient does not increase the patient's incidence of major bleeding by more than 0.5%, 0.75%, 1%, 1.25% or 1.5%.

It will be understood that the use of approximate or about, when referring to either an amount of milvexian (or pharmaceutically acceptable salt thereof) or time interval for administering milvexian (or pharmaceutically acceptable salt thereof) may be an amount or time interval that is within 5% (greater or less than), or within 7.5% (greater or less than), or within 10% (greater or less than), or within 12.5% (greater or less than), or within 15% (greater or less than), or within 17.5% (greater or less than), or within 20% (greater or less than) any specified amount or time interval.

The disclosure is also directed to the following aspects:

Aspect 1. A method of treating or preventing a venous thromboembolic disorder comprising: orally administering to a patient in need thereof a total daily dose of from 25 mg to 400 mg of milvexian, or pharmaceutically acceptable salt thereof.

Aspect 2. The method according to aspect 1, wherein the total daily dose is from 50 mg to 400 mg.

Aspect 3. The method according to aspect 1, wherein the total daily dose is 25 mg.

Aspect 4. The method according to aspect 1, wherein the total daily dose is 50 mg.

Aspect 5. The method according to aspect 1, wherein the total daily dose is 100 mg.

Aspect 6. The method according to aspect 1, wherein the total daily dose is 150 mg.

Aspect 7. The method according to aspect 1, wherein the total daily dose is 200 mg.

Aspect 8. The method according to any one of aspects 1 to 7, wherein the total daily dose is administered in a single dose.

Aspect 9. The method according to any one of aspects 1 to 7, wherein the total daily dose is administered in divided doses.

Aspect 10. The method according to aspect 9, wherein the total daily dose is administered in two doses.

Aspect 11. The method according to any one of aspects 1 to 10, wherein the venous thromboembolic disorder is proximal and/or distal deep vein thrombosis, nonfatal pulmonary embolism, or death.

Aspect 12. The method according to aspect 11, wherein the venous thromboembolic disorder is proximal and/or distal deep vein thrombosis.

Aspect 13. The method according to aspect 11, wherein the venous thromboembolic disorder is nonfatal pulmonary embolism.

Aspect 14. The method according to aspect 11, wherein the venous thromboembolic disorder is death.

Aspect 15. A method of preventing post-operative venous thromboembolism events in a patient recovering from surgery, said method comprising administering to said patient a daily dose of milvexian, or pharmaceutically acceptable salt thereof, wherein said administration results in the patient experiencing fewer post-operative venous thromboembolism events than would result from subcutaneous administration to the patient of 40 mg per day of enoxaparin.

Aspect 16. The method according to aspect 15, wherein the patient is recovering from abdominal surgery, knee replacement surgery, or hip replacement surgery.

Aspect 17. The method according to aspect 15 or aspect 16, wherein the daily dose of milvexian, or pharmaceutically acceptable salt thereof, is 50 mg.

Aspect 18. The method according to aspect 15 or aspect 16, wherein the daily dose of milvexian, or pharmaceutically acceptable salt thereof, is 100 mg.

Aspect 19. The method according to aspect 15 or aspect 16, wherein the daily dose of milvexian, or pharmaceutically acceptable salt thereof, is 150 mg.

Aspect 20. The method according to aspect 15 or aspect 16, wherein the daily dose of milvexian, or pharmaceutically acceptable salt thereof, is 200 mg.

Aspect 21. The method according to any one of aspects 15 to 20, wherein the daily dose of milvexian, or pharmaceutically acceptable salt thereof, is administered in a single dose.

Aspect 22. The method according to any one of aspects 15 to 20, wherein the daily dose of milvexian, or pharmaceutically acceptable salt thereof, is administered in divided doses.

Aspect 23. A method of treating or preventing a venous thromboembolic disorder comprising: orally administering therapeutically effective amount of milvexian, or pharmaceutically acceptable salt thereof once or twice daily to a patient in need thereof, wherein an incidence rate of total venous thromboembolism events in the patient treated with the milvexian, or pharmaceutically acceptable salt thereof is less than 25% with an alpha of 5%.

Aspect 24. The method of aspect 23, wherein the incidence rate of total venous thromboembolism events is less than 20%.

Aspect 25. The method of aspect 23, wherein the incidence rate of total venous thromboembolism events is less than 15%.

Aspect 26. The method of aspect 23, wherein the incidence rate of total venous thromboembolism events is less than 10%.

Aspect 27. The method of aspect 23, wherein the milvexian, or pharmaceutically acceptable salt thereof is administered at a total daily dose ranging from 50 mg to 400 mg.

Aspect 28. The method of aspect 23, wherein the milvexian, or pharmaceutically acceptable salt thereof is administered at the total daily dose selected from the group consisting of 50 mg, 100 mg, 200 mg, and 400 mg.

Aspect 29. The method of aspect 23, wherein the milvexian, or pharmaceutically acceptable salt thereof is administered at the total daily dose of 50 mg.

Aspect 30. The method of aspect 23, wherein the milvexian, or pharmaceutically acceptable salt thereof is administered at the total daily dose of 100 mg.

Aspect 31. The method of aspect 23, wherein the milvexian, or pharmaceutically acceptable salt thereof is administered at the total daily dose of 150 mg.

Aspect 32. The method of aspect 23, wherein the milvexian, or pharmaceutically acceptable salt thereof is administered at the total daily dose of 200 mg.

Aspect 33. The method of aspect 23, wherein the milvexian, or pharmaceutically acceptable salt thereof is administered at the total daily dose of 400 mg.

Aspect 34. The method of any one of aspects 23 to 33, wherein milvexian is administered twice daily.

Aspect 35. The method of any one of aspects 23 to 33, wherein milvexian is administered once daily.

Aspect 36. The method of any one of aspects 23 to 35, wherein the rate of venous thromboembolism occurs in a dose dependent manner without increasing the risk of bleeding compared with enoxaparin.

Aspect 37. The method of any one of aspects 23 to 36, wherein the therapeutically effective amount is clinically proven effective in achieving less than 25% of the incidence rate of total venous thromboembolism events in the patient treated with milvexian, or pharmaceutically acceptable salt thereof.

Aspect 38. Use of an oral dosage form of milvexian, or pharmaceutically acceptable salt thereof, for manufacture of a medicament for treating or preventing a venous thromboembolic disorder by orally administering to a patient in need thereof a total daily dose of from 25 mg to 400 mg of milvexian, or pharmaceutically acceptable salt thereof.

Aspect 39. The use according to aspect 38, wherein the total daily dose is from 50 mg to 400 mg.

Aspect 40. The use according to aspect 38, wherein the total daily dose is 25 mg.

Aspect 41. The use according to aspect 38, wherein the total daily dose is 50 mg.

Aspect 42. The use according to aspect 38, wherein the total daily dose is 100 mg.

Aspect 43. The use according to aspect 38, wherein the total daily dose is 150 mg.

Aspect 44. The use according to aspect 38, wherein the total daily dose is 200 mg.

Aspect 45. The use according to any one of aspects 38 to 44, wherein the total daily dose is administered in a single dose.

Aspect 46. The use according to any one of aspects 38 to 44, wherein the total daily dose is administered in divided doses.

Aspect 47. The use according to aspect 46, wherein the total daily dose is administered in two doses.

Aspect 48. The use according to any one of aspects 38 to 47, wherein the venous thromboembolic disorder is proximal and/or distal deep vein thrombosis, nonfatal pulmonary embolism, or death.

Aspect 49. The use according to aspect 48, wherein the venous thromboembolic disorder is proximal and/or distal deep vein thrombosis.

Aspect 50. The use according to aspect 48, wherein the venous thromboembolic disorder is nonfatal pulmonary embolism.

Aspect 51. The use according to aspect 48, wherein the venous thromboembolic disorder is death.

Aspect 52. Use of an oral dosage form of milvexian, or pharmaceutically acceptable salt thereof, for manufacture of a medicament for preventing post-operative venous thromboembolism events in a patient recovering from surgery, wherein administration of said medicament to said patient results in the patient experiencing fewer post-operative venous thromboembolism events than would result from subcutaneous administration to the patient of 40 mg per day of enoxaparin.

Aspect 53. The use according to aspect 52, wherein the patient is recovering from abdominal surgery, knee replacement surgery, or hip replacement surgery.

Aspect 54. The use according to aspect 52 or aspect 53, wherein the daily dose of milvexian, or pharmaceutically acceptable salt thereof, is 50 mg.

Aspect 55. The use according to aspect 52 or aspect 53, wherein the daily dose of milvexian, or pharmaceutically acceptable salt thereof, is 100 mg.

Aspect 56. The use according to aspect 52 or aspect 53, wherein the daily dose of milvexian, or pharmaceutically acceptable salt thereof, is 150 mg.

Aspect 57. The use according to aspect 52 or aspect 53, wherein the daily dose of milvexian, or pharmaceutically acceptable salt thereof, is 200 mg.

Aspect 58. The use according to any one of aspects 52 to 57, wherein the daily dose of milvexian, or pharmaceutically acceptable salt thereof, is administered in a single dose.

Aspect 59. The use according to any one of aspects 52 to 57, wherein the daily dose of milvexian, or pharmaceutically acceptable salt thereof, is administered in divided doses.

Aspect 60. Use of a solid dosage form of milvexian, or pharmaceutically acceptable salt thereof, for manufacture of a medicament adapted for once or twice daily administration to a patient for treating or preventing a venous thromboembolic disorder wherein said administration results in an incidence rate of total venous thromboembolism events in the patient that is less than 25% with an alpha of 5%.

Aspect 61. The use of aspect 60, wherein the incidence rate of total venous thromboembolism events is less than 20%.

Aspect 62. The use of aspect 60, wherein the incidence rate of total venous thromboembolism events is less than 15%.

Aspect 63. The use of aspect 60, wherein the incidence rate of total venous thromboembolism events is less than 10%.

Aspect 64. The use of aspect 60, wherein the milvexian, or pharmaceutically acceptable salt thereof is administered at a total daily dose ranging from 50 mg to 400 mg.

Aspect 65. The use of aspect 60, wherein the milvexian, or pharmaceutically acceptable salt thereof is administered at the total daily dose selected from the group consisting of 50 mg, 100 mg, 200 mg, and 400 mg.

Aspect 66. The use of aspect 60, wherein the milvexian, or pharmaceutically acceptable salt thereof is administered at the total daily dose of 50 mg.

Aspect 67. The use of aspect 60, wherein the milvexian, or pharmaceutically acceptable salt thereof is administered at the total daily dose of 100 mg.

Aspect 68. The use of aspect 60, wherein the milvexian, or pharmaceutically acceptable salt thereof is administered at the total daily dose of 150 mg.

Aspect 69. The use of aspect 60, wherein the milvexian, or pharmaceutically acceptable salt thereof is administered at the total daily dose of 200 mg.

Aspect 70. The use of aspect 60, wherein the milvexian, or pharmaceutically acceptable salt thereof is administered at the total daily dose of 400 mg.

Aspect 71. The use of any one of aspects 60 to 70, wherein milvexian is administered twice daily.

Aspect 72. The use of any one of aspects 60 to 70, wherein milvexian is administered once daily.

Aspect 73. The use of any one of aspects 60 to 72, wherein the rate of venous thromboembolism occurs in a dose dependent manner without increasing the risk of bleeding compared with enoxaparin.

Aspect 74. The use of any one of aspects 60 to 73, wherein the therapeutically effective amount is clinically proven effective in achieving less than 25% of the incidence rate of total venous thromboembolism events in the patient treated with milvexian, or pharmaceutically acceptable salt thereof.

Those skilled in the art will recognize, or be able to ascertain using no more than routine experimentation, many equivalents to the embodiments described herein.

All publications, patents and patent applications mentioned in this specification are herein incorporated by reference into the specification to the same extent as if each individual publication, patent or patent application was specifically and individually indicated to be incorporated herein by reference.

### EXAMPLES

### Example 1

A Study of milvexian versus subcutaneous enoxaparin in participants undergoing elective total knee replacement surgery was conducted as follows.

The study was a randomized, open-label, study drug-dose blind, multicenter study to evaluate the efficacy and safety of milvexian, an oral factor XIa inhibitor, versus subcutaneous enoxaparin in subjects undergoing elective total knee replacement surgery.

The purpose of this study is to determine the efficacy of milvexian in preventing total venous thromboembolism (VTE) events (proximal and/or distal deep vein thrombosis [DVT][asymptomatic confirmed by venography assessment or objectively confirmed symptomatic], nonfatal pulmonary embolism [PE], or any death) during the treatment period.

| | |
|---|---|
| Study Type | Interventional |
| Study Phase | Phase 2 |
| Study | Allocation: Randomized |
| Design | Intervention Model: Parallel Assignment |
| | Masking: Single (Outcomes Assessor) |
| | Masking Description: |
| | Treatment arms and study drug dose regimens will be blinded. |
| | Primary Purpose: Prevention |
| Condition | Arthroplasty, Replacement, Knee |
| Intervention | Drug: milvexian 25 mg |
| | Participants will receive milvexian 25 mg (1*25 mg capsule) |
| | BID (in Group A) or once daily (in Group E), orally for 10 to 14 |
| | postoperative days. |
| | Drug: milvexian 50 mg |
| | Participants will receive milvexian 50 mg (2*25 mg capsules) |
| | BID orally for 10 to 14 postoperative days. |
| | Drug: milvexian 100 mg |
| | Participants will receive milvexian 100 mg (1*100 mg capsule) |
| | BID, orally for 10 to 14 postoperative days. |
| | Drug: milvexian 200 mg |
| | Participants will receive milvexian 200 mg (2*100 mg capsules) |
| | BID (in Group D) or once daily (in Group F), orally for 10 to 14 |
| | postoperative days. |
| | Drug: Placebo |
| | Participants will receive placebo matching to milvexian, orally. |
| | Drug: enoxaparin 40 mg |
| | Participants will receive enoxaparin 40 mg once daily |
| | subcutaneously for 10 to 14 postoperative days. |
| Study Arms | Experimental: Group A: milvexian 25 mg + Placebo BID |
| | Participants will receive milvexian 25 milligram (mg) (1*25 mg |
| | capsule) and 1 placebo capsule twice daily (BID), orally for 10 to |
| | 14 postoperative days. |
| | Interventions: |
| | Drug: milvexian 25 mg |
| | Drug: Placebo |
| | Experimental: Group B: milvexian 50 mg BID |
| | Participants will receive milvexian 50 mg (2*25 mg capsules) |
| | BID orally for 10 to 14 postoperative days. |
| | Intervention: Drug: milvexian 50 mg |
| | Experimental: Group C: milvexian 100 mg + Placebo BID |
| | Participants will receive milvexian 100 mg (1*100 mg capsule) |
| | and 1 placebo capsule BID orally for 10 to 14 postoperative days. |
| | Interventions: |
| | Drug: milvexian 100 mg |
| | Drug: Placebo |
| | Experimental: Group D: milvexian 200 mg BID |
| | Participants will receive milvexian 200 mg (2*100 mg capsules) |
| | BID orally for 10 to 14 postoperative days. |
| | Intervention: Drug: milvexian 200 mg |
| | Experimental: Group E: milvexian 25 mg Once Daily + Placebo |
| | Participants will receive milvexian 25 mg (1*25 mg capsule) |
| | once daily and 1 placebo capsule in the morning and 2 placebo |
| | capsules in the evening, orally for 10 to 14 postoperative days. |
| | Interventions: |
| | Drug: milvexian 25 mg |
| | Drug: Placebo |
| | Experimental: Group F: milvexian 200 mg Once Daily + Placebo |
| | Participants will receive milvexian 200 mg (2*100 mg capsules |
| | in the morning) once daily and 2 placebo capsules in the evening, |
| | orally for 10 to 14 postoperative days. |
| | Interventions: |
| | Drug: milvexian 200 mg |
| | Drug: Placebo |
| | Experimental: Group G: milvexian 50 mg once daily + Placebo |
| | Participants will receive milvexian 50 mg (2*25 mg capsules in |
| | the morning) once daily and 2 placebo capsules in the evening, |
| | orally for 10 to 14 postoperative days. |
| | Interventions: |
| | Drug: milvexian 50 mg |
| | Drug: Placebo |
| | Active Comparator: Group I: enoxaparin 40 mg Once Daily |
| | Participants will receive enoxaparin 40 mg once daily |
| | subcutaneously for 10 to 14 postoperative days. |
| | Intervention: Drug: enoxaparin 40 mg |
| Enrollment | 1242 |
| Eligibility | Inclusion Criteria: |
| Criteria | Medically stable and appropriate for anticoagulant prophylaxis as |
| | determined by the investigator on the basis of physical |
| | examination, medical history, and vital signs performed as part of |
| | screening for elective total knee replacement (TKR) surgery |
| | Medically stable and appropriate for anticoagulant prophylaxis on |
| | the basis of clinical laboratory tests performed as part of local |
| | standard-of-care as part of screening for elective TKR surgery |
| | Has plans to undergo an elective primary unilateral TKR surgery |
| | A woman must be- a) Not of childbearing potential; b) Of |
| | childbearing potential and practicing a highly effective method of |
| | contraception (failure rate of less than [<]1 percent [%] per year |
| | when used consistently and correctly) and agrees to remain on a |
| | highly effective method for the duration of study drug with |
| | milvexian plus 5 half-lives of study drug plus 30 days (duration of |
| | ovulatory cycle) for a total of 34 days after the completion of |
| | treatment, pregnancy testing (serum or urine) prior to the first |
| | dose of study drug |
| | Willing and able to adhere to the lifestyle restrictions specified in |
| | this protocol |
| | Exclusion Criteria: |
| | History of any condition for which the use of low molecular- |
| | weight heparin (LMWH) is not recommended in the opinion of |
| | the investigator (for example, previous allergic reaction, |
| | creatinine clearance <30 milliliter per minute [mL/minute]) |
| | History of severe hepatic impairment |
| | Planned bilateral revision or unicompartmental procedure |
| | Unable to undergo venography (for example, due to contrast agent |
| | allergy, poor venous access, or impaired renal function that would |
| | increase the risk of contrast-induced nephropathy |
| | Known previous pulmonary embolism (PE) or deep vein |
| | thrombosis (DVT) in either lower extremity |
| Sex/Gender | Sexes Eligible for Study: All |
| Ages | 50 Years and older (Adult, Older Adult) |
| Accepts | No |
| Healthy | |
| Volunteers | |
| Primary | Number of Participants with Total Venous Thromboembolism (VTE) |
| Outcome | [Time Frame: Up to 14 days] |
| Measures | Total VTE is defined as the composite of proximal and/or distal Deep |
| | Vein Thrombosis (DVT) (asymptomatic confirmed by venography |
| | assessment of the operated leg or objectively confirmed symptomatic), |
| | nonfatal pulmonary embolism (PE), or any death. |
| Secondary | Number of Participants with any Bleeding Event |
| Outcome | [Time Frame: Up to 14 days] |
| Measures | Any bleeding event is defined as the composite of major, |
| | clinically relevant nonmajor, and/or minimal bleeding events. |
| | Number of Participants with Total VTE [Time Frame: Up to |
| | Week 6] |
| | Total VTE is defined as the composite of proximal and/or distal |
| | Deep Vein Thrombosis (DVT) (asymptomatic confirmed by |
| | venography assessment of the operated leg or objectively |
| | confirmed symptomatic), nonfatal pulmonary embolism (PE), or |
| | any death. |
| | Number of Participants with any Bleeding Event |
| | [Time Frame: Up to Week 6] |
| | Any bleeding event is defined as the composite of major, |
| | clinically relevant nonmajor, and/or minimal bleeding events. |
| | Number of Participants with Composite of Major and Clinically |
| | Relevant Nonmajor Bleeding Events [Time Frame: Up to Week |
| | 6] |
| | Bleeding events composite of major and clinically relevant |
| | nonmajor events will be assessed through Week 6. |
| | Number of Participants with Major Bleeding Events |
| | [Time Frame: Up to Week 6] |
| | Major bleeding is defined as: Fatal bleeding; bleeding that is |
| | symptomatic and occurs in a critical area or organ and/or; |
| | extrasurgical site bleeding causing a fall in hemoglobin level of |
| | 20 gram per liter (g/L) (1.24 millimole per liter [mmol/L]) or |
| | more, or leading to transfusion of 2 or more units of whole blood |
| | or red cells with temporal association within 24-48 hours to the |
| | bleeding, and/or; surgical site bleeding that requires a second |
| | intervention open, arthroscopic, endovascular, or a hemarthrosis |
| | resulting in prolonged hospitalization or a deep wound infection |
| | and/or; surgical site bleeding that is unexpected and prolonged |
| | and/or sufficiently large to cause hemodynamic instability. |
| | Number of Participants with Clinically Relevant Nonmajor |
| | Bleeding Events [Time Frame: Up to Week 6] |
| | Clinically relevant nonmajor bleeding is defined as acute |
| | clinically overt bleeding which does not satisfy additional criteria |
| | required for the bleeding event to be defined as a major bleeding |
| | event and meets at least 1 of the following criteria: Epistaxis |
| | (nose bleed), Gastrointestinal bleed, Hematuria, |
| | Bruising/ecchymosis, Hemoptysis, Hematoma. |
| | Number of Participants with Minimal Bleeding Events |
| | [Time Frame: Up to Week 6] |
| | Any overt bleeding not meeting major or clinically relevant |
| | nonmajor criteria will be assessed as minimal bleeding. |
| | Number of Participants with Major VTE [Time Frame: Up to 14 |
| | days and up to Week 6] |
| | Major VTE is a composite of asymptomatic or symptomatic |
| | proximal DVT, PE, or any death. |
| | Number of Participants with Proximal and/or Distal Deep Vein |
| | Thrombosis (DVT) [Time Frame: Up to 14 days and up to Week |
| | 6] |
| | Number of Participants with Proximal and/or Distal Deep Vein |
| | Thrombosis (DVT) will be assessed. DVT asymptomatic |
| | confirmed by venography assessment of the operated leg or |
| | objectively confirmed symptomatic. |
| | Number of Participants with Nonfatal Pulmonary Embolism (PE) |
| | [Time Frame: Up to 14 days and up to Week 6] |
| | Number of Participants with Nonfatal Pulmonary Embolism (PE) |
| | will be assessed. For all participants with symptoms of PE, spiral |
| | computed tomography (CT), pulmonary angiography, or |
| | perfusion/ventilation lung scintigraphy combined with chest |
| | radiography will be performed. |
| | Number of Participants with Deaths [Time Frame: Up to 14 days |
| | and up to Week 6] |
| | Number of participants with deaths will be reported. |
| | Apparent Clearance (CL/F) of milvexian [Time Frame: Day 1: 2, |
| | 4, and 12 hours postdose; Day 2; Day 4: 0, 2, 4, and 12 hours |
| | postdose; Day 7; and Day 10 to Day 14] |
| | Clearance of a drug is a measure of the rate at which a drug is |
| | metabolized or eliminated by normal biological processes. |
| | Clearance obtained after oral dose (apparent oral clearance) is |
| | influenced by the fraction of the dose absorbed. |
| | Apparent Volume of Distribution (V/F) of milvexian |
| | [Time Frame: Day 1: 2, 4, and 12 hours postdose; Day 2; Day 4: |
| | 0, 2, 4, and 12 hours postdose; Day 7; and Day 10 to Day 14] |
| | V/F is defined as the theoretical volume in which the total |
| | amount of drug would need to be uniformly distributed to |
| | produce the desired serum concentration of a drug. |
| | Impact of Selected Demographics or Laboratory Values |
| | (Example [e.g.] sex, age, Weight) on Apparent Clearance (CL/F) |
| | [Time Frame: Day 1: 2, 4, and 12 hours postdose; Day 2; Day 4: |
| | 0, 2, 4, and 12 hours postdose; Day 7; and Day 10 to Day 14] |
| | Impact of selected demographics or laboratory values (e.g. sex, |
| | age, weight) on Apparent Clearance (CL/F) will be assessed. |
| | Impact of Selected Demographics or Laboratory Values (e.g. sex, |
| | age, Weight) on Apparent Volume of Distribution (V/F) |
| | [Time Frame: Day 1: 2, 4, and 12 hours postdose; Day 2; Day 4: |
| | 0, 2, 4, and 12 hours postdose; Day 7; and Day 10 to Day 14] |
| | Impact of selected demographics or laboratory values (e.g. sex, |
| | age, weight) on V/F will be assessed. |
| | Relationship between milvexian Dose Levels with Total VTE |
| | [Time Frame: Up to 14 days] |
| | Relationship between milvexian dose levels with total VTE will |
| | be determined using a multiple comparison procedures and |
| | modeling (MCP-Mod) approach. |
| | Relationship Between milvexian Dose Levels with Composite of |
| | Major or Clinically Relevant Nonmajor Bleeding Events |
| | [Time Frame: Up to 14 days] |
| | Relationship between milvexian dose levels with composite of |
| | major or clinically relevant nonmajor bleeding events will be |
| | determined using a MCP-Mod approach. |
| | Relationship between milvexian Dose Levels with Major |
| | Bleeding Events [Time Frame: Up to 14 days] |
| | Relationship between milvexian dose levels with major bleeding |
| | events will be determined using a MCP-Mod approach. |
| | Relationship between milvexian Dose Levels with Clinically |
| | Relevant Nonmajor Bleeding Events [Time Frame: Up to 14 |
| | days] |
| | Relationship between milvexian dose levels with clinically |
| | relevant nonmajor bleeding events will be determined using a |
| | MCP-Mod approach. |
| | Relationship between milvexian Dose Levels with Minimal |
| | Bleeding Events [Time Frame: Up to 14 days] |
| | Relationship between milvexian dose levels with minimal |
| | bleeding events will be determined using a MCP-Mod approach. |
| | Relationship Between milvexian Exposure with Total VTE |
| | [Time Frame: Up to 14 days] |
| | Relationship between milvexian exposure with total VTE, |
| | determined using Exposure-Response analysis. |
| | Relationship Between milvexian Exposure with Composite of |
| | Major or Clinically Relevant Nonmajor Bleeding Events |
| | [Time Frame: Up to 14 days] |
| | Relationship between milvexian exposure with composite of |
| | major or clinically relevant nonmajor bleeding events, |
| | determined using Exposure-Response analysis. |
| Secondary | Number of Participants with any Bleeding Event |
| Outcome | [Time Frame: Up to 14 days] |
| Measures | Any bleeding event is defined as the composite of major, |
| | clinically relevant nonmajor, and/or minimal bleeding events. |
| | Number of Participants with Total VTE [Time Frame: Up to |
| | Week 6] |
| | Total VTE is defined as the composite of proximal and/or distal |
| | Deep Vein Thrombosis (DVT) (asymptomatic confirmed by |
| | venography assessment of the operated leg or objectively |
| | confirmed symptomatic), nonfatal pulmonary embolism (PE), or |
| | any death. |
| | Number of Participants with any Bleeding Event |
| | [Time Frame: Up to Week 6] |
| | Any bleeding event is defined as the composite of major, |
| | clinically relevant nonmajor, and/or minimal bleeding events. |
| | Number of Participants with Composite of Major and Clinically |
| | Relevant Nonmajor Bleeding Events [Time Frame: Up to Week |
| | 6] |
| | Bleeding events composite of major and clinically relevant |
| | nonmajor events will be assessed through Week 6. |
| | Number of Participants with Major Bleeding Events |
| | [Time Frame: Up to Week 6] |
| | Major bleeding is defined as: Fatal bleeding; bleeding that is |
| | symptomatic and occurs in a critical area or organ and/or; |
| | extrasurgical site bleeding causing a fall in hemoglobin level of |
| | 20 gram per liter (g/L) (1.24 millimole per liter [mmol/L]) or |
| | more, or leading to transfusion of 2 or more units of whole blood |
| | or red cells with temporal association within 24-48 hours to the |
| | bleeding, and/or; surgical site bleeding that requires a second |
| | intervention open, arthroscopic, endovascular, or a hemarthrosis |
| | resulting in prolonged hospitalization or a deep wound infection |
| | and/or; surgical site bleeding that is unexpected and prolonged |
| | and/or sufficiently large to cause hemodynamic instability. |
| | Number of Participants with Clinically Relevant Nonmajor |
| | Bleeding Events [Time Frame: Up to Week 6] |
| | Clinically relevant nonmajor bleeding is defined as acute |
| | clinically overt bleeding which does not satisfy additional criteria |
| | required for the bleeding event to be defined as a major bleeding |
| | event and meets at least 1 of the following criteria: Epistaxis |
| | (nose bleed), Gastrointestinal bleed, Hematuria, |
| | Bruising/ecchymosis, Hemoptysis, Hematoma. |
| | Number of Participants with Minimal Bleeding Events |
| | [Time Frame: Up to Week 6] |
| | Any bleeding event not meeting major or clinically relevant |
| | nonmajor criteria will be recorded as minimal bleeding event. |
| | Number of Participants with Major VTE [Time Frame: Up to 14 |
| | days and up to Week 6] |
| | Major VTE is a composite of asymptomatic or symptomatic |
| | proximal DVT, PE, or any death. |
| | Number of Participants with Proximal and/or Distal Deep Vein |
| | Thrombosis (DVT) [Time Frame: Up to 14 days and up to Week |
| | 6] |
| | Number of Participants with Proximal and/or Distal Deep Vein |
| | Thrombosis (DVT) will be assessed. DVT asymptomatic |
| | confirmed by venography assessment of the operated leg or |
| | objectively confirmed symptomatic. |
| | Number of Participants with Nonfatal Pulmonary Embolism (PE) |
| | [Time Frame: Up to 14 days and up to Week 6] |
| | Number of Participants with Nonfatal Pulmonary Embolism (PE) |
| | will be assessed. For all participants with symptoms of PE, spiral |
| | computed tomography (CT), pulmonary angiography, or |
| | perfusion/ventilation lung scintigraphy combined with chest |
| | radiography will be performed. |
| | Number of Participants with Deaths [Time Frame: Up to 14 days |
| | and up to Week 6] |
| | Number of participants with deaths will be reported. |
| | Apparent Clearance (CL/F) of milvexian [Time Frame: Day 1: 2, |
| | 4, and 12 hours postdose; Day 2; Day 4: 0, 2, 4, and 12 hours |
| | postdose; Day 7; and Day 10 to Day 14] |
| | Clearance of a drug is a measure of the rate at which a drug is |
| | metabolized or eliminated by normal biological processes. |
| | Clearance obtained after oral dose (apparent oral clearance) is |
| | influenced by the fraction of the dose absorbed. |
| | Apparent Volume of Distribution (V/F) of milvexian |
| | [Time Frame: Day 1: 2, 4, and 12 hours postdose; Day 2; Day 4: |
| | 0, 2, 4, and 12 hours postdose; Day 7; and Day 10 to Day 14] |
| | V/F is defined as the theoretical volume in which the total |
| | amount of drug would need to be uniformly distributed to |
| | produce the desired serum concentration of a drug. |
| | Impact of Selected Demographics or Laboratory Values |
| | (Example [e.g.] sex, age, Weight) on Apparent Clearance (CL/F) |
| | [Time Frame: Day 1: 2, 4, and 12 hours postdose; Day 2; Day 4: |
| | 0, 2, 4, and 12 hours postdose; Day 7; and Day 10 to Day 14] |
| | Impact of selected demographics or laboratory values (e.g. sex, |
| | age, weight) on Apparent Clearance (CL/F) will be assessed. |
| | Impact of Selected Demographics or Laboratory Values (e.g. sex, |
| | age, Weight) on Apparent Volume of Distribution (V/F) |
| | [Time Frame: Day 1: 2, 4, and 12 hours postdose; Day 2; Day 4: |
| | 0, 2, 4, and 12 hours postdose; Day 7; and Day 10 to Day 14] |
| | Impact of selected demographics or laboratory values (e.g. sex, |
| | age, weight) on V/F will be assessed. |
| | Relationship between milvexian Dose Levels with Total VTE |
| | [Time Frame: Up to 14 days] |
| | Relationship between milvexian dose levels with total VTE will |
| | be determined using a multiple comparison procedures and |
| | modeling (MCP-Mod) approach. |
| | Relationship Between milvexian Dose Levels with Composite of |
| | Major or Clinically Relevant Nonmajor Bleeding Events |
| | [Time Frame: Up to 14 days] |
| | Relationship between milvexian dose levels with composite of |
| | major or clinically relevant nonmajor bleeding events will be |
| | determined using a MCP-Mod approach. |
| | Relationship between milvexian Dose Levels with Major |
| | Bleeding Events [Time Frame: Up to 14 days] |
| | Relationship between milvexian dose levels with major bleeding |
| | events will be determined using a MCP-Mod approach. |
| | Relationship between milvexian Dose Levels with Clinically |
| | Relevant Nonmajor Bleeding Events [Time Frame: Up to 14 |
| | days] |
| | Relationship between milvexian dose levels with clinically |
| | relevant nonmajor bleeding events will be determined using a |
| | MCP-Mod approach. |
| | Relationship between milvexian Dose Levels with Minimal |
| | Bleeding Events [Time Frame: Up to 14 days] |
| | Relationship between milvexian dose levels with minimal |
| | bleeding events will be determined using a MCP-Mod approach. |
| | Relationship Between milvexian Exposure with Total VTE |
| | [Time Frame: Up to 14 days] |
| | Relationship between milvexian exposure with total VTE, |
| | determined using Exposure-Response analysis. |
| | Relationship Between milvexian Exposure Levels with |
| | Composite of Major or Clinically Relevant Nonmajor Bleeding |
| | Events [Time Frame: Up to 14 days] |
| | Relationship between milvexian exposure levels with composite |
| | of major or clinically relevant nonmajor bleeding events, |
| | determined using Exposure-Response analysis. |

This study was a dose guiding study for the direct factor XIa inhibitor milvexian. The objective of the study was the assessment of safety, tolerability and efficacy of milvexian at different oral doses (twice daily (BID or bid) and once daily (QD or qd)) compared with subcutaneously administered enoxaparin 40 mg in the prevention of venous thromboembolism.

### Methods

The study is a Phase 2, open-label, double-blinded, randomized, active-controlled, multicenter dose-ranging study of milvexian in subjects undergoing primary elective unilateral total knee replacement (TKR) surgery. The study used a prospective, randomized, open-label, blinded endpoint (PROBE) design.

1242 subjects were randomized. Patients were randomized into treatment groups that either included milvexian or enoxaparin. The characteristics of the subjects in the milvexian and enoxaparin groups were balanced and typical for subjects in similar studies.

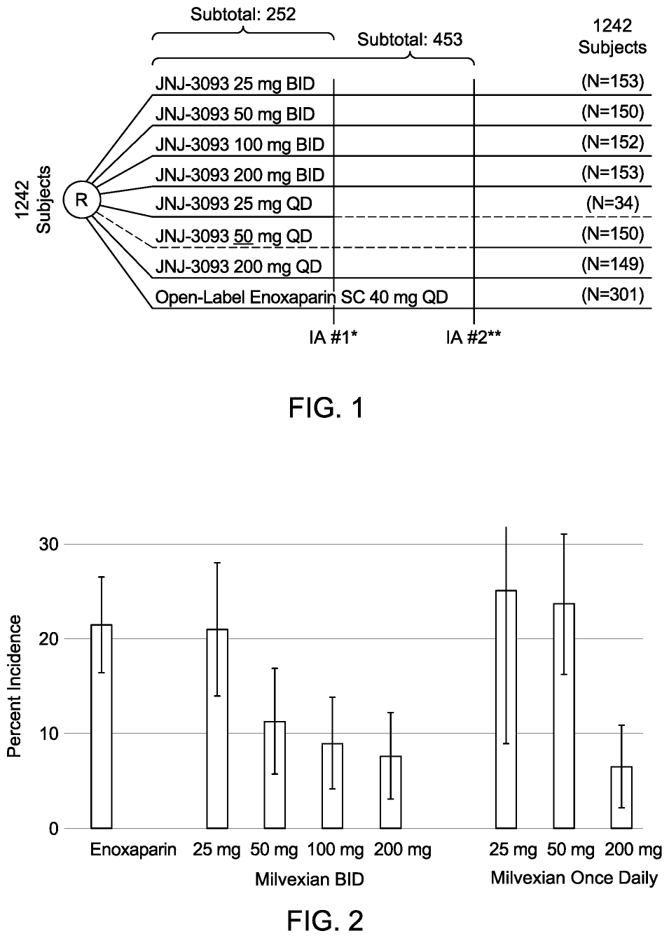

There were a total of 8 treatment arms over the life of the study: 4 milvexian BID dose regimens ranging from 25 to 200 BID, 3 milvexian once daily dose regimens of 25, 50, 200 mg, and an enoxaparin 40 mg once daily control arm. The 4 milvexian BID dose regimens, and the milvexian 200 mg once daily arm were continued uninterrupted enabling each arm to randomize 150 subjects. The enoxaparin control arm likewise completed its pre-specified randomization of 300 subjects. The milvexian 25 mg once daily dose-regimen was halted by an unblinded Operations Committee (OC), responsible for reviewing ongoing safety and efficacy data by subject treatment assignments approximately every 3 to 8 weeks, during an ad hoc interim analysis (IA); enrollment in the 25 mg once daily milvexian treatment arm was truncated at 34 subjects. At that time, an amendment to the protocol allowed for the initiation of a milvexian 50 mg once daily regimen. Under a subsequent recommendation of the OC, the randomization ratio was modified enabling the milvexian 50 mg once daily arm to achieve it full complement of 150 subjects randomized. FIG. 1 provides the study design, including number of subjects randomized into each study arm.

The study consisted of three phases: an up to 30-day screening phase before TKR surgery, a 10 to 14 day postoperative dosing phase (mean duration of treatment for both the milvexian- and enoxaparin-treated groups was 11.7 days) and a 4-week follow up phase. The first dose of milvexian was administered 12 to 24 hours post-surgery which was defined as beginning at wound closure.

### Patients

Patients undergoing elective unilateral total knee arthroplasty were eligible if they were 50 years of age or older, had plans to undergo an elective primary unilateral TKR surgery, and medically stable and appropriate candidates for anticoagulant prophylaxis on the basis of clinical laboratory tests performed as part of local standard-of-care as part of screening for elective TKR surgery. The main exclusion criteria were contraindications to enoxaparin (e.g., creatinine clearance <30 mL/minute), a history of severe hepatic impairment or prior venous thromboembolism, a requirement for chronic antithrombotic therapy other than aspirin (100 mg per day or less), or the inability to undergo venography.

### Randomization and Study Treatment

An adaptive design was used to optimize patient recruitment for the dose-response evaluation. The number of dose regimens, the option to implement additional once daily dosing regimens, and the randomization ratio were based on the results of the periodic reviews and interim analysis. Initially, eligible patients were randomly assigned, in a 1:1:1:1:1:1:2 ratio, to one of seven parallel treatment groups, which included four twice daily milvexian regimens (25 mg, 50 mg, 100 mg, or 200 mg), two once daily milvexian regimens (25 mg or 200 mg), and enoxaparin, respectively (FIG. 1). Randomization was performed postoperatively using a centralized interactive web-based response system and was stratified by geographic region of the study centers.

Oral milvexian or matching placebo was started 12 to 24 hours after surgery. Enoxaparin was given subcutaneously at a dose of 40 mg once daily and was started either the evening before or 12 to 24 hours after surgery. Treatment with milvexian or enoxaparin was given for 10 to 14 days after surgery.

### Study Outcomes

The primary efficacy outcome was venous thromboembolism, defined as the composite of asymptomatic deep-vein thrombosis (detected by mandatory unilateral venography performed 10 to 14 days after surgery), confirmed symptomatic venous thromboembolism (symptomatic deep-vein thrombosis of the leg or nonfatal pulmonary embolism), or death. Unilateral venography performed only on the operated leg detects over 90% of deep-vein thrombosis in patients undergoing unilateral knee arthroplasty and reduces the risk for the patient (Buller et al. Factor XI antisense oligonucleotide for prevention of venous thrombosis. N Engl J Med 2015; 372:232-40; Fuji et al., A dose-ranging study evaluating the oral factor Xa inhibitor edoxaban for the prevention of venous thromboembolism in patients undergoing total knee arthroplasty. J Thromb Haemost 2010; 8:2458-68).

The major secondary efficacy outcomes were: (a) proximal deep-vein thrombosis (symptomatic or asymptomatic), (b) distal deep-vein thrombosis (symptomatic or asymptomatic) (c) nonfatal pulmonary embolism, and (d) death. An exploratory efficacy outcome was the extent of venous thrombosis on venography, which was assessed by the adjudication committee using predefined categories.

The principal safety outcome was any bleeding, which was defined as the composite of major, clinically relevant nonmajor, and minimal bleeding. Secondary safety outcomes were: (a) major bleeding, (b) clinically relevant nonmajor bleeding, (c) clinically relevant bleeding, defined as the composite of major and clinically relevant nonmajor bleeding, and (d) minimal bleeding. Bleeding was classified as major if it was overt and associated with a decrease in hemoglobin of 2 g/dL or more or necessitated transfusion of 2 or more units of blood with a temporal association within 24 to 48 hours of the bleed; or if it occurred in a critical area or organ or contributed to death. Bleeding at the surgical site was defined as major only if it required intervention; caused hemodynamic instability; or caused hemarthrosis that delayed mobilization or wound healing and resulted in prolonged hospitalization or deep wound infection. Overt bleeding not meeting the criteria for major bleeding, but that necessitated medical examination or intervention or had clinical consequences, was classified as clinically relevant nonmajor bleeding. Bleeding not meeting the criteria for major or clinically relevant nonmajor bleeding was classified as minimal bleeding (Schulman et al., Definition of major bleeding in clinical investigations of antihemostatic medicinal products in surgical patients. J Thromb Haemost 2010; 8:202-4).

### Surveillance and Follow-Up

Patients were evaluated preoperatively within 30 days of surgery, at the time of randomization, and after surgery on days 1, 4, 7, and 10 to 14, and at 6 weeks (±10 days). Patients were instructed to report symptoms suggestive of venous thromboembolism or bleeding.

### Laboratory Measurements

The activated partial-thromboplastin time and prothrombin time were measured in a central laboratory using Actin FS and Innovin, respectively (Siemens Healthcare, Tarrytown, NY). Activated partial-thromboplastin time and prothrombin time ratios were calculated by dividing postoperative values by those measured preoperatively. The primary efficacy outcome focused on twice-daily milvexian regimens because with a half-life of about 11 hours, milvexian is suitable for twice daily dosing.

### Statistical Analysis

Proof-of-efficacy is defined as either a statistically significant dose-response or a primary endpoint event rate for the combined BID milvexian groups that is statistically lower than 30% with 95% confidence interval (95% CI, one-sided alpha of 5%) from the network meta-analysis. Data from the network meta-analysis estimated the placebo incidence rate of total VTE to be 50% (95% CI, 40, 60). For the purposes of the study, a more conservative incidence rate of 30% (the 95% upper bound for enoxaparin in the network meta-analysis) was chosen.

The primary analysis for efficacy was performed in the modified intention-to-treat population, which included all patients who received at least one dose of study medication and had an evaluable venogram within the prespecified time window, a documented symptomatic venous thromboembolic event, or a fatal event. The rate of venous thromboembolism in the combined milvexian twice-daily dose groups was compared with 30% using the binomial test Evidence for a dose-response trend with the twice-daily milvexian regimens was evaluated with the MCP-Mod framework using pre-specified models (Pinheiro et al., Model-based dose finding under model uncertainty using general parametric models. Statistics in Medicine 2014; 33:1646-61). For analysis of the major secondary efficacy outcomes, the risk ratio, and the corresponding confidence interval for each milvexian group relative to enoxaparin, were calculated using the Cochran-Mantel-Haenszel method with study region as a stratification factor.

Analysis of safety outcomes was performed in the safety population, which included all randomized patients who received at least one dose of study medication; the time for analysis included the on-treatment period plus 2 days. For each bleeding outcome, the rate in each milvexian group was compared with that in the enoxaparin group with the same methods used for evaluation of the secondary efficacy outcomes. The Kaplan-Meier method was used to assess the time to the first occurrence of any bleeding with milvexian or enoxaparin.

### Results

### Participants

A total of 1242 subjects were randomized and they were all included in the ITT analysis set. Of the 1242 subjects, 1219 (98.1%) subjects received at least 1 dose of study drug, therefore in the safety analysis set. Moreover, 1048 (84.4%) subjects were included in the mITT-CEC (Day 14) analysis set. In the ITT analysis set, 1230 (99.0%) subjects completed the study, and 12 (1.0%) subjects prematurely discontinued the study (11 [0.9%] due to withdrawal by subject and 1 [0.1%] due to death). In the safety analysis set, 1162 (95.3%) subjects completed the study treatment, and 57 (4.7%) subjects prematurely discontinued the study treatment (35 [2.9%] due to adverse event, 16 [1.3%] due to subject refused further study treatment, 1 [0.1%] due to death, and 5 [0.4%] due to other). As shown in Table 1, in the ITT analysis set, 366 (29.5%) of subjects were male, and 1081 (87.5%) subjects were white. The mean (SD) of age was 68.0 (8.02), and the median of age was 68.0. Demographic and baseline characteristics were balanced among the groups. The baseline characteristics are presented in FIG. 1 and Table 1.

TABLE 1

| | Baseline Characteristics | | | | | | | | | |
|---|---|---|---|---|---|---|---|---|---|---|
| | 25 mg | 50 mg | 25 mg | 50 mg | 200 mg | 100 mg | 200 mg | | | |
| | QD | QD | BID | BID | QD | BID | BID | Combined | ENOX | Total |
| Modified | 34 | 150 | 153 | 150 | 149 | 152 | 153 | 941 | 301 | 1242 |
| intention-to-treat | | | | | | | | | | |
| population(ITT): | | | | | | | | | | |
| N | | | | | | | | | | |
| Age, years | | | | | | | | | | |
| N | 34 | 150 | 153 | 150 | 149 | 152 | 153 | 941 | 301 | 1242 |
| Mean (SD) | 68.1 | 67.9 | 68.4 | 68.8 | 68.0 | 67.0 | 68.6 | 68.1 | 67.8 | 68.0 |
| | (5.74) | (8.03) | (8.49) | (8.17) | (8.25) | (8.00) | (7.76) | (8.04) | (7.97) | (8.02) |
| 50 -< 65 years | 9 | 51 | 47 | 43 | 51 | 55 | 49 | 305 | 104 | 409 |
| | (26.5%) | (34.0%) | (30.7%) | (28.7%) | (34.2%) | (36.2%) | (32.0%) | (32.4%) | (34.6%) | (32.9%) |
| >=65 years | 25 | 99 | 106 | 107 | 98 | 97 | 104 | 636 | 197 | 833 |
| | (73.5%) | (66.0%) | (69.3%) | (71.3%) | (65.8%) | (63.8%) | (68.0%) | (67.6%) | (65.4%) | (67.1%) |
| Sex | | | | | | | | | | |
| N | 34 | 150 | 153 | 150 | 149 | 152 | 153 | 941 | 301 | 1242 |
| Female | 24 | 107 | 113 | 108 | (72.5%) | 102 | 106 | 668 | 208 | 876 |
| | (70.6%) | (71.3%) | (73.9%) | (72.0%) | 108 | (67.1%) | (69.3%) | (71.0%) | (69.1%) | (70.5%) |
| Male | 10 | 43 | 40 | 42 | 41 | 50 | 47 | 273 | 93 | 366 |
| | (29.4%) | (28.7%) | (26.1%) | (28.0%) | (27.5%) | (32.9%) | (30.7%) | (29.0%) | (30.9%) | (29.5%) |
| Body mass | | | | | | | | | | |
| index, kg/m² | | | | | | | | | | |
| N | 33 | 150 | 150 | 149 | 145 | 148 | 148 | 923 | 295 | 1218 |
| Mean (SD) | 31.7 | 30.4 | 31.5 | 30.4 | 31.6 | 31.4 | 31.4 | 31.2 | 30.7 | 31.0 |
| | (6.94) | (5.67) | (5.29) | (5.89) | (5.65) | (5.22) | (5.86) | (5.66) | (5.70) | (5.67) |
| Underweight | 0 | 0 | 0 | 1 | 0 | 1 (0.7%)\|1 | (0.7%)3 | (0.3%) | 2 (0.7%) | 5 (0.4%) |
| <18.5 | | | | (0.7%) | | | | | | |
| Duration of | | | | | | | | | | |
| surgery-hr | | | | | | | | | | |
| Median | 1.4 | 1.4 | 1.3 | 1.4 | 1.4 | 1.4 | 1.4 | | 1.4 | |
| Range | 0.8-2.8 | 0.6-3.3 | 0.5-3.5 | 0.7-3.1 | 0.7-3.5 | 0.5-6.6 | 0.6-2.8 | | 0.4-3.5 | |
| Time after | | | | | | | | | | |
| surgery to | | | | | | | | | | |
| ambulation- | | | | | | | | | | |
| days | | | | | | | | | | |
| Median | 1 | 1 | 1 | 1 | 1 | 1 | 1 | | 1 | |
| Range | 0-4 | 0-4 | 0-3 | 0-3 | 0-6 | 0-4 | 0-4 | | 0-4 | |
| Baseline | | | | | | | | | | |
| activated partial- | | | | | | | | | | |
| thromboplastin | | | | | | | | | | |
| time-sec | | | | | | | | | | |
| Median | 26 | 26 | 26 | 27 | 26 | 26 | 26 | | 26 | |
| Interquartile | 25-28 | 25-29 | 24-28 | 25-29 | 25-28 | 25-28 | 24-28 | | 25-28 | |
| Range | | | | | | | | | | |
| Safety population | 33 | 150 | 148 | 148 | 147 | 149 | 148 | | 296 | |
| No. of patients | | | | | | | | | | |
| Time after | | | | | | | | | | |
| surgery to | | | | | | | | | | |
| milvexian or | | | | | | | | | | |
| enoxaparin | | | | | | | | | | |
| administration- | | | | | | | | | | |
| hr | | | | | | | | | | |
| Median | 22 | 22 | 22 | 22 | 23 | 22 | 22 | | 22 | |
| Range | 13-25 | 13-26 | 13-26 | 13-26 | 14-26 | 13-25 | 12-26 | | 7-34 | |
| Duration of | | | | | | | | | | |
| milvexian or | | | | | | | | | | |
| enoxaparin | | | | | | | | | | |
| treatment-days | | | | | | | | | | |
| Median | 12 | 13 | 12 | 12 | 12 | 12 | 12 | | 12 | |
| Range | 9-14 | 6-16 | 1-16 | 2-16 | 1-16 | 4-17 | 4-16 | | 1-15 | |

(i). Mean age was comparable between combined milvexian and enoxaparin ((68.1 vs 67.8 years).
(ii). Approximately 2/3 of subjects were ≥65 years old, 67.6% in combined milvexian and 65.4% in enoxaparin
(iii). Most subjects were female, 71% in combined milvexian and 69.1% in enoxaparin
(iv). Mean BMI was comparable between combined milvexian (31.2 kg/m2) and enoxaparin (30.7 kg/m2) .
(v). Most subjects had BMI ≥30 kg/m2, 54.4% in milvexian and 49.5% in enoxaparin. Milvexian 25 mg BID and 100 mg BID had slightly higher rate of BMI ≥30 kg/m2, 62.0% and 60.8.
(vi). The normal range for the activated partial-thromboplastin time is 22 to 29 sec.

### Efficacy—Prevention of Total Venous Thromboembolism Events (VTE)

Evaluable venogramns were obtained in 1,047 of the 1219 patients (86%) who received study medication (FIG. 1). This study provides proof-of-concept that milvexian is an effective antithrombotic agent. See. e.g., Ting et al., Phase II clinical development of new drugs. pp. iv-v, 5-10 New York: Springer; 2017. Milvexian significantly reduced the rate of venous thromboembolism after elective knee arthroplasty in a dose-dependent manner with both twice-daily and once-daily regimens. The current data indicate that postoperative milvexian provides effective thromboprophylaxis against venous thromboembolism.

Efficacy outcomes are provided in Table 2. In the twice daily milvexian groups, the primary efficacy outcome occurred in 27 of 129 patients (21.0%), in 14 of 124 patients (11.3%), in 12 of 134 patients (9.0%/), and in 10 of 131 patients (7.6%) given 25 mg, 50 mg, 100 mg, or 200 mg of milvexian (total daily milvexian doses of 50 mg, 100 mg, 200 mg and 400 mg), respectively (Table 2); findings consistent with a statistically significant dose response (P=0.0004 (one-sided)). The primary efficacy outcome occurred in 63 of 518 patients (12.2%) given twice daily milvexian; a rate significantly lower (P<0.0001 (one-sided)) than the prespecified benchmark of 30%. Thus, both proof-of-efficacy criteria were met.

There was a statistically significant (P=0.0003 (one-sided)) dose-response for the primary efficacy outcome with the once-daily milvexian regimens (Table 2).

The primary objective of the study was to determine the efficacy of milvexian in preventing total VTE. Total VTE was defined as proximal and/or distal deep vein thrombosis ((DVT); asymptomatic confirmed by venography assessment or objectively confirmed symptomatic), nonfatal pulmonary embolism (PE), or any death during the 10-14 day treatment period. Proof-of efficacy was defined as either a statistically significant dose-response or a primary endpoint event rate for the combined BID milvexian groups that was statistically lower than 30%. The study met this primary objective of total VTE for the pooled milvexian BID dose regimens compared to the prespecified target of 30% (with statistically significant reduction in total VTE (p<0.0001 (one-sided))).

Of the 1242 randomized subjects in the trial, 1048 (84%) had an evaluable venogram performed to assess occurrence of VTE. Of those 1048 subjects, e.g., the “analysis set”, total VTE was reported in 108 (13.6%) of 796 subjects who received milvexian (all milvexian dose/dose regimen arms combined), 63 (12.2%) of 518 subjects who received a milvexian BID dose regimen, and 54 (21.4%) of 252 subjects who received enoxaparin (relative risk ratio 0.64 (95% CI 0.48-0.85) for combined milvexian vs. enoxaparin; relative risk ratio 0.57 (95% CI 0.41-0.79) for milvexian BID versus enoxaparin).

TABLE 2
Primary Efficacy Event and Major VTE Component - Relative Risk and
Confidence Interval for Comparison Between Milvexian and Enoxaparin (Enox). CEC
Adjudicated, mITT, at Day 14.

| | | Milvexian | | | | | | |
|---|---|---|---|---|---|---|---|---|
| | | BID Dose Regimen | | | | Once Daily Dose Regimen | | |
| Outcomes | Enox | 25 mg | 50 mg | 100 mg | 200 mg | 25 mg | 50 mg | 200 mg |
| Number of | 252 | 129 | 124 | 134 | 131 | 28 | 127 | 123 |
| patients | | | | | | | | |
| evaluated | | | | | | | | |
| Total VTE | 54 | 27 | 14 | 12 | 10 | 7 | 30 | 8 |
| n (%) | (21.4%) | (20.9%) | (11.3%) | (9.0%) | (7.6%) | (25.0%) | (23.6%) | (6.5%) |
| RR | | 0.97 | 0.53 | 0.42 | 0.37 | 1.00 | 1.15 | 0.30 |
| (95% CI) vs. | | (0.65- | (0.31- | (0.23- | (0.19- | (0.51- | (0.78- | (0.15- |
| Enoxaparin | | 1.45) | 0.90) | 0.76) | 0.69) | 1.97) | 1.70) | 0.62) |
| Major VTE n | 4 | 1 | 1 | 2 | 0 | 0 | 2 | 0 |
| (%) | (1.6%) | (0.8%) | (0.8%) | (1.5%) | | | (1.6%) | |

Key: RR = Relative Risk Ratio;
CI = 95% Confidence Interval of the incidence rate;
Cochran-Mantel-Haenszel method with region as a stratification factor was used for the calculation of RR;
VTE = Venous thromboembolism;
DVT-Deep vein thrombosis.

As illustrated in FIG. 2 and Table 2, for total VTE, statistically significant dose responses were seen across the 4 milvexian BID regimens (p=0.0004 (one-sided)) and the 3 once daily dosing regimens (p=0.0003 (one-sided)). Statistically significant reductions in VTE occurred with the 50 mg, 100 mg and 200 mg BID and 200 QD dosing regimens versus enoxaparin, while doses of 25 mg BID and 25 mg and 50 mg once daily performed similarly to enoxaparin. Of note, none of the milvexian dose arms which spanned an 8-fold daily dose range appeared less effective than enoxaparin. Events of major VTE were infrequent and occurred in 4 subjects (1.6%) in the enoxaparin group, 1 subject (0.8%) in the 25 mg BID milvexian group, 1 subject (0.8%) in the 50 mg BID milvexian group, 2 subjects (1.5%) in 100 mg BID milvexian group, and 2 subjects (1.6%) in the 50 mg once daily milvexian group. As there were few major VTE events, (e.g., proximal DVT, PE and death), the majority of total VTE events were minor, e.g., distal, DVTs. See Table 3, below.

TABLE 3
Total VTE events, by event type, per clinical events committee, through day 14

| | | Milvexian | | | | |
|---|---|---|---|---|---|---|
| | | Combined | BID Regimen | | | |
| | Enoxaparin | BID | 25 mg | 50 mg | 100 mg | 200 mg |
| | N = 252 | N = 518 | N = 129 | N = 124 | N = 134 | N = 131 |
| | n (%) | n (%) | n (%) | n (%) | n (%) | n (%) |
| Total VTE | 54 (21.4) | 63 (12.2) | 27 (20.9) | 14 (11.3) | 12 (9.0) | 10 (7.6) |
| Proximal DVT | 1 (0.4) | 2 (0.4) | 1 (0.8) | 0 | 1 (0.7) | 0 |
| Asymptomatic | 1 (0.4) | 2 (0.4) | 1 (0.8) | 0 | 1 (0.7) | 0 |
| proximal DVT | | | | | | |
| Symptomatic | 0 | 0 | 0 | 0 | 0 | 0 |
| proximal DVT | | | | | | |
| Non-fatal PE | 1 (0.4) | 2 (0.4) | 0 | 1 (0.8) | 1 (0.7) | 0 |
| Death | 1 (0.4) | 0 | 0 | 0 | 0 | 0 |
| Distal DVT | 50 (19.8) | 59 (11.4) | 26 (20.2) | 13 (10.5) | 10 (7.5) | 10 (7.6) |
| Asymptomatic | 50 (19.8) | 59 (11.4) | 26 (20.2) | 13 (10.5) | 10 (7.5) | 10 (7.6) |
| distal DVT | | | | | | |
| Symptomatic distal DVT | 0 | 1 (0.2) | 0 | 0 | 1 (0.7) | 0 |

Overall, the dose data indicated that milvexian at total daily doses (TDDs) of ≥100 mg significantly reduced total VTE events when compared to enoxaparin, and that at TDDs of ≥200 mg milvexian significantly reduced total VTE by >50% when compared to enoxaparin. Milvexian showed comparable VTE incidence rates when compared to enoxaparin at the lowest completed dose groups (once daily 25 mg, once daily 50 mg and 25 mg BID).

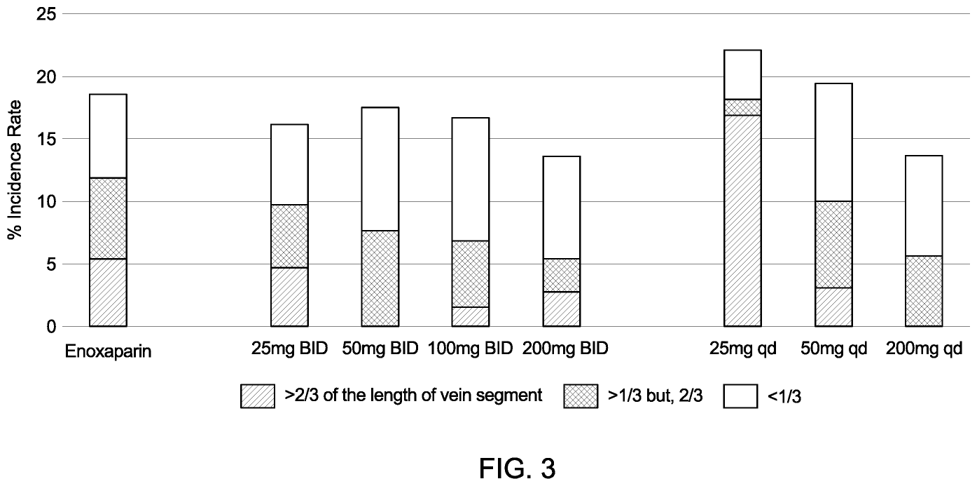

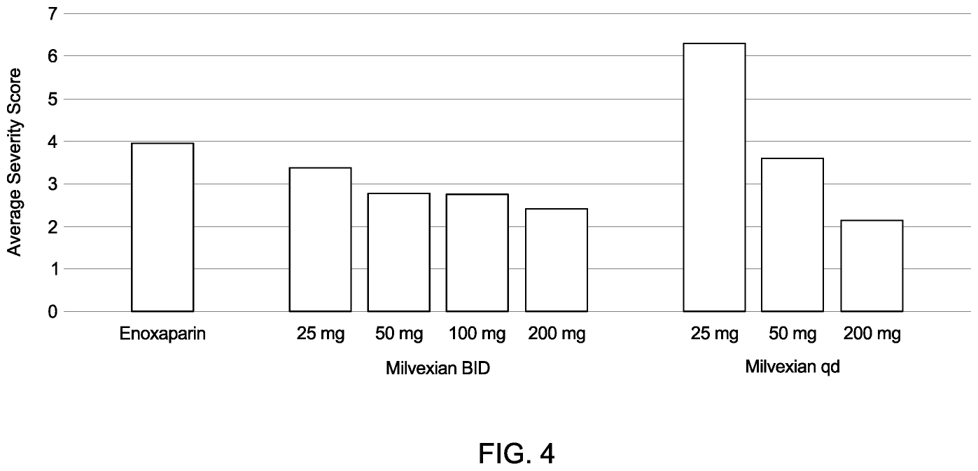

Milvexian at TDDs of ≥100 mg not only significantly reduced the number of total VTE events compared to enoxaparin, it also significantly reduced severity of DVT, the largest contributor to the number of total VTE events, in subjects with DVT. Venograms, assessed by independent blinded adjucators, were used to identify, and to assign DVT severity scores to, subjects with DVT. For each assessed venogram, 11 segments were evaluated for the presence and extent of clot. For each evaluated segment, a severity score was assigned depending on the presence and, if present, extension of the clot in the segment of the vein. Severity scores, on a scale of 0-3, were assigned as follows: 0=no clot; 1=less than ⅓; 2=greater than or equal to ⅓ but less than ⅔; and 3=greater than ⅔ the length of the vein segment. As illustrated in FIG. 3, the incidence of segments with the most severe score of 3 in the subjects treated with milvexian at TDDs of ≥100 mg (% incidence rates of 0, 1.52, 2.73 and 0 for subjects in the 50 mg BID, 100 mg BID, 200 mg BID and 200 mg once daily milvexian arms, respectively) was less than half that of the subjects treated with enoxaparin (5.42% incidence rate). Moreover, surprisingly, subjects treated with either 50 mg BID milvexian or 200 mg once daily milvexian had a 0% incidence rate of DVT with severity score of 3. Overall, the average severity score of subjects with DVT on venography was 1.17 to 1.81 lower in the subjects treated with milvexian at TDDs of 2100 mg relative to those treated with enoxaparin. See FIG. 4.

### Safety—Occurrence of Bleeding

The key secondary objective of the study was to assess the dose-response trend of milvexian for the occurrence of Any Bleeding events during the treatment period. Any Bleeding was defined as the composite of major bleeding according to the International Society on Thrombosis and Haemostasis (ISTH) criteria modified for the surgical setting, clinically relevant nonmajor bleeding events or minimal bleeding events as assessed by the clinical events committee (CEC). The rates of venous thromboembolism were significantly lower with daily milvexian doses of 100 mg or more than that with enoxaparin. Although the rate of any bleeding was 4.1% with both milvexian and enoxaparin, the rate of the composite of major and clinically relevant nonmajor bleeding was low and there was no evidence of a dose response with a 16-fold range of total daily milvexian doses from 25 mg to 400 mg. Therefore, postoperative milvexian is effective for preventing venous thromboembolism and is associated with a low risk of clinically relevant bleeding.

The bleeding outcomes are provided in Table 4. The principal safety outcome of any bleeding occurred in 38 of 923 patients (4.1%) given milvexian and in 12 of 296 patients (4.1%) given enoxaparin. Most bleeds were in the minimal category and involved the surgical site. There were no major bleeds with milvexian and 1 with enoxaparin. Rates of clinically relevant nonmajor bleeding with milvexian and enoxaparin were 0.8% and 1.4%, respectively.

Incidence rates of Any Bleeding across the 8-fold milvexian dose range and for enoxaparin were low, occurring in only 50 subjects. For comparator enoxaparin, the overall incidence rate of bleeding was less than half the rates anticipated on the basis of an internally conducted network meta-analysis (4.1% observed versus 7.22% anticipated); network meta-analysis of published results involving the various anticoagulants in the setting of prevention of VTE following TKR (Weitz et al. Milvexian for Prevention of Venous Thromboembolism. NEJM. 2021; protocol). See Table 4.

TABLE 4
Expected event rates for total VTE, Any Bleeding, and major
+ CRNM bleeding by treatment using a network meta-analysis

| | Event Rate: Median (95% CI) | | |
|---|---|---|---|
| Treatment | Total VTE | Any Bleeding | Major + CRNM Bleed |
| Placebo | 50.0% [40, 60] | 4.54% [3.5, 5.9] | 2.20% [1.7, 2.8] |
| Enoxaparin | 23.8% [18, 30] | 7.22% [6.0, 8.8] | 3.55% [3.0, 4.2] |
| 40 mg QD | | | |

*Any bleeding is a composite ISTH major bleeding in a surgical setting, clinically relevant nonmajor bleeding (CRNM) events, or minimal bleeding events

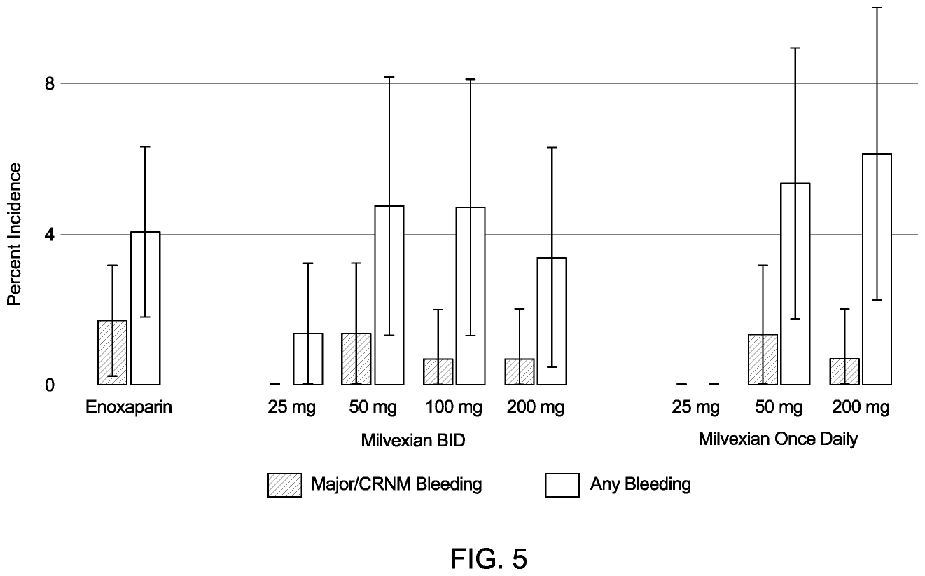

With milvexian, Any Bleeding ranged from 0% for subjects randomized to 25 mg once daily to 6.1% at the 200 mg once daily regimen. The 25 mg once daily or BID dosing was associated with little bleeding (0%, 1.4% once daily, BID respectively). The incidence of Any Bleeding increased with higher milvexian dosing ranging from 4.7% (50 mg or 100 mg BID) to 6.1% (200 mg QD). However, there was no dose response when assessed b total daily dose administered. See FIG. 5 and Table 5.

TABLE 5
Relative Risk and Confidence Interval for the Comparison of On-Treatment Principal
Safety Endpoint and Components Major+CRNM and Minimal Bleeding Events (CEC
Adjudicated) between Milvexian Arms and Enoxaparin Arm

| | | Milvexian | | | | | | |
|---|---|---|---|---|---|---|---|---|
| | | BID Dose Regimen | | | | Once Daily Dose Regimen | | |
| | Enox | 25 mg | 50 mg | 100 mg | 200 mg | 25 mg | 50 mg | 200 mg |
| Analysis set | 296 | 148 | 148 | 149 | 148 | 33 | 159 | 147 |
| Any Bleeding | 12 | 2 | 7 | 7 | 5 | 0 | 8 | 9 |
| n (%) | (4.1%) | (1.4%) | (4.7%) | (4.7%) | (3.4%) | | (5.3%) | (6.1%) |
| RR (95% CI) | | 0.33 | 1.15 | 1.14 | 0.81 | — | 1.17 | 1.51 |
| vs Enox | | (0.08- | (0.47- | (0.47- | (0.29- | | (0.50- | (0.66- |
| | | 1.43) | 2.82) | 2.80) | 2.24) | | 2.72) | 3.43) |
| Major+CRNM | 5 | 0 | 2 | 1 | 1 | 0 | 2 | 1 |
| n (%) | | | (1.4%) | (0.7%) | (0.7%) | | (1.3%) | (0.7%) |
| RR (95% CI) | (1.7%) | — | 0.79 | 0.39 | 0.39 | | 0.68 | 0.40 |
| vs Enox | | | (0.16- | (0.05- | (0.05- | | (0.14- | (0.05- |
| | | | 3.96 | 3.30) | 3.28) | | 3.39) | 3.34) |
| Minimal | 8 | 2 | 5 | 7 | 4 | 0 | 6 | 8 |
| Bleeding | (2.7%) | (1.4%) | (3.4%) | (4.7%) | (2.7%) | | (4.0%) | (5.4%) |
| RR (95% CI) | | 0.50 | 1.23 | 1.71 | 0.96 | | 1.35 | 2.01 |
| vs Enox | | (0.11- | (0.42- | (0.64- | (0.30- | — | (0.48- | (0.78- |
| | | 2.26) | 3.65) | 4.58) | 3.12) | | 3.75) | 5.19) |

Key: RR = Relative Risk Ratio.
CI = 95% Confidence Interval of the incidence rate;
Cochran-Mantel-Haenszel method with region as a stratification factor was used for the calculation of RR.
Note:
On-Treatment period is defined as from the initial administration of the study drug through the day of lastdose + 2 days.
Source output: TSFMOD02C

ISTH major+CRNM bleeding incidence rate across the 8-fold milvexian dose range and for enoxaparin were lower than expected for a study of this size and duration, occurring in a combined total of only 12 subjects, but provided a more clinically relevant and objective assessment of the milvexian bleeding profile. Major+CRNM bleeding was reported in 5 (1.7%) of subjects treated with enoxaparin, approximately half the 3.55% historic incidence rate in a VTE setting. There were no fatal bleeds and only one major bleeding event in a subject who presented with a sub-dural hematoma while taking enoxaparin. Major+CRNM bleeding was reported in 7 (0.8%) subjects treated with milvexian, which provided incidence rates of 0, 1.4%, 0.7%, 0.7%, 1.3% and 0.7% for milvexian 25, 50, 100, 200 mg BID, 50 and 200 mg QD, respectively, e.g., an incidence range of 0.7% to 1.4%. Milvexian was not associated with any overt bleeding liability with no fatal or major bleeding events. Incidence of any bleeding was comparable between enoxaparin and milvexian (combined) for 3 bleeding classifications (major, CRNM, minimal) (Table 6). No CEC adjudicated major bleeding in milvexian group (1 in enoxaparin). Incidence of CRNM bleeding was higher for enoxaparin however numerically low for both groups. Majority of bleeding is skin related, no trends toward increased bleeding in areas of high fibrinolytic activity (e.g., oral cavity, nose, and urinary tract, etc.) in milvexian group. Numerically more minimal bleeding events were reported with milvexian (all combined) than enoxaparin. The higher incidence was driven by the skin bleeding site with the majority reported around the operative site.

TABLE 6
Incidence Rate and Relative Risk for the Comparison of Principal
Safety Endpoint and Components (CEC Adjudicated) between Milvexian
Arms and Enoxaparin Arm; Safety Analysis Set/On Treatment.

| | | Milvexian | Milvexian |
|---|---|---|---|
| | Enoxaparin | Combined | BID |
| | N = 296 | N = 923 | N = 593 |
| Any | 12 (4.1) | 38 (4.1) | 21 (3.5) |
| bleeding, | | 0.99 (0.53-1.85) | 0.86 (0.43-1.71) |
| n (%) | | | |
| RR (CI) | | | |
| Major | 1 (0.3) | 0 | 0 |
| Bleeding, | | | |
| n (%) | | | |
| RR (CI) | | | |
| Clinically | 4 (1.4) | 7 (0.8) | 4 (0.7) |
| relevant | | 0.55 (0.16-1.85) | 0.49 (0.12-1.96) |
| non-major | | | |
| Bleeding, | | | |
| n (%) | | | |
| RR (CI) | | | |
| Major bleeding | 5 (1.7) | 7 (0.8) | 4 (0.7) |
| or CRNM | | 0.44 (0.14-1.35) | 0.39 (0.11-1.44) |
| Bleeding, | | | |
| n (%) | | | |
| RR (CI) | | | |
| Minimal | 8 (2.7) | 32 (3.5) | 18 (3.0) |
| Bleeding, | | 1.26 (0.59-2.67) | 1.10 (0.49-2.49) |
| n (%) | | | |
| RR (CI) | | | |

Overall, no dose response of the bleeding incidence for milvexian groups; no incremental bleeding risk observed at increased doses, the bleeding incidence with milvexian was low and plateaued across all doses tested. This is in contradistinction to all approved anticoagulants, including direct oral anticoagulants (DOACs) (e.g., apixaban, rivaroxaban, etc.), where higher rates of bleeding are seen with increasing exposure.

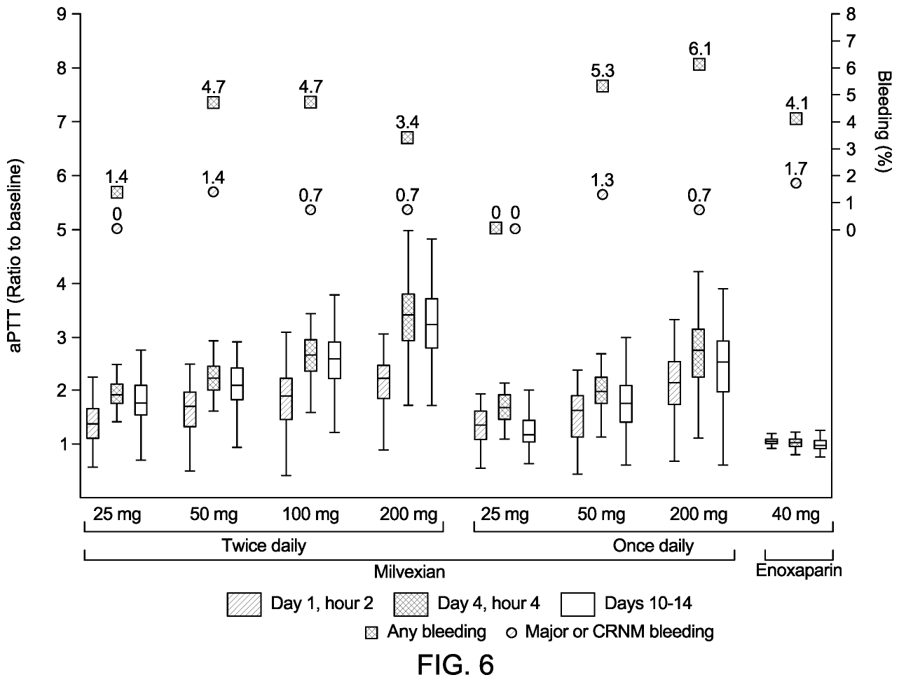

Milvexian treatment increased the activated partial-thromboplastin time ratio in a dose-dependent manner, whereas enoxaparin had no apparent effect. No evidence of a dose-dependent increase with respect to bleeding was noted with milvexian (FIG. 6). Neither milvexian nor enoxaparin increased the prothrombin time ratio. Median activated partial-thromboplastin time (aPTT) ratios and rates of any and clinically relevant bleeding (defined as the composite of major and clinically relevant nonmajor bleeding) with the various doses of milvexian and with enoxaparin are shown in FIG. 6. In the activated partial-thromboplastin time ratio plots, the middle line indicates the median; the top and bottom of the box indicate the upper and lower limits, respectively, of the interquartile range; and the vertical lines above and below the box indicate the upper and lower limits, respectively, of the range. The squares indicate rates of any bleeding, while the circles indicate the rates of clinically relevant bleeding, the composite of major and clinically relevant nonmajor bleeding. aPTT denotes activated partial-thromboplastin time.

### Example 2

Milvexian was evaluated for prevention and treatment of thrombosis with a rabbit model of electrically induced carotid artery thrombus (ECAT) in rabbits. The rabbit ECAT model has been calibrated against clinical results in VTE prevention based on apixaban. In this model, targeting the concentration that led to 50% reduction in thrombus weight was correlated with the steady-state trough concentration of the clinical dose of apixaban.

The rabbit ECAT model as described by Wong et al., Nonpeptide factor Xa inhibitors: II. Antithrombotic evaluation in a rabbit model of electrically induced carotid artery thrombosis. J Pharmacol Exp Ther. 2000; 295: 212-8, was used in this study to assess the effects of vehicle and milvexian on carotid blood flow after thrombus induction.

Male New Zealand white rabbits were anesthetized with ketamine (50 mg/kg+50 mg/kg/h intramuscularly) and xylazine (10 mg/kg+10 mg/kg/h intramuscularly). Thrombosis was induced by electrical stimulation of the control carotid artery for 3 minutes at 4 mA using an external stainless-steel bipolar electrode. Carotid blood flow was measured with an electromagnetic flow probe continuously over a 90-minute period to monitor thrombosis induced occlusion. Integrated carotid blood flow was measured by the area under the flowtime curve (see Wong et al.). In addition, thrombus from the injured artery was removed, blotted twice on a weighing paper to remove residual fluid, and weighed.

To achieve a stable plasma level with minimum experimental variability, compounds were administered by IV infusion. Thrombosis was then electrically induced in the opposite carotid artery, using the same method as mentioned previously. In the prevention ECAT model, milvexian or its vehicle was given by a bolus injection supplemented with a continuous IV infusion 30 minutes prior to the vessel injury. In the treatment ECAT model (see Wong P C, et al., BMS-593214, an active site-directed factor VIIa inhibitor: enzyme kinetics. antithrombotic and antihaemostatic studies. Thromb Haemost. 2010; 104: 261-9), milvexian and vehicle were given as stated previously at 15 minutes after the vessel injury. The IV infusion was continued throughout the experiment for both models. Concentrations of milvexian in plasma samples were measured by a specific and sensitive liquid chromatographic/mass spectrometry method (LC/MS/MS).

In the prevention study, the milvexian group consisted of vehicle (10% N,N dimethylacetamide:25% PEG300:65% of 5% dextrose) and milvexian (mg/kg+mg/kg/h) at 0.063+0.04, 0.25+0.17, and 1+0.67 (n=6 per group). In the treatment study, the milvexian group consisted of vehicle (10% N,N dimethylacetamide:25% PEG300:65% of 5% dextrose; n=6) and milvexian (mg/kg+mg/kg/h) at 0.25+0.17 and 1+0.67 (n=6 per group).

In the prevention ECAT study, plasma concentrations of milvexian at 30 and 120 minutes after the start of the infusion were determined. The average value of plasma concentrations of milvexian at 30 and 120 minutes after the start of infusion in each animal was then used for the analysis of the concentration-response curve and the determination of antithrombotic EC₅₀ (the concentration that gives 50% maximal response). Integrated blood flow, thrombus weight, aPTT (activated partial thromboplastin time), TT (thrombin time), and PT (prothrombin time) were also measured. In the treatment ECAT study, only the plasma concentrations of milvexian at the end of the study were determined.

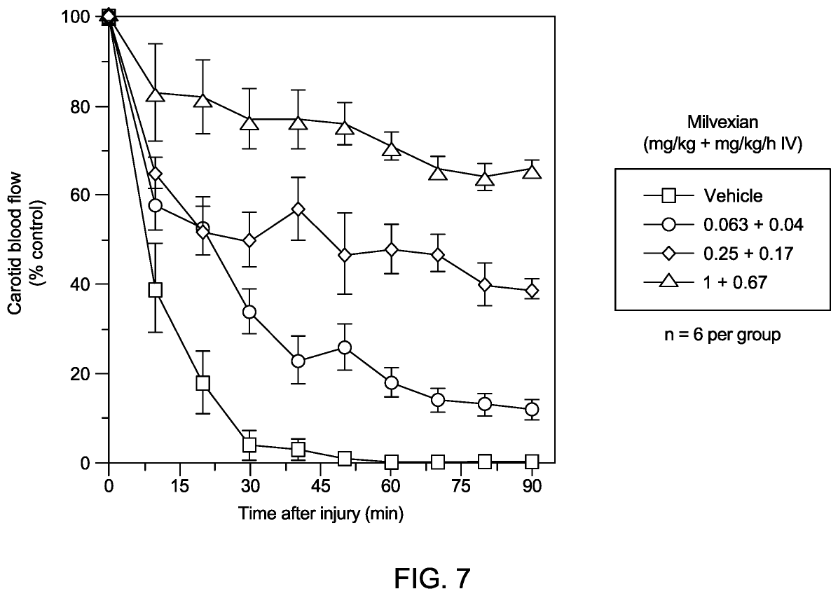

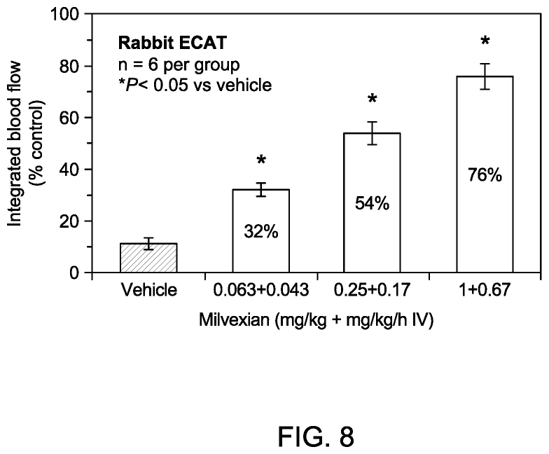

FIG. 7 shows the effects of vehicle and milvexian on carotid blood flow after thrombus induction in the prevention ECAT rabbit model. Following electric current stimulation, thrombus formation was induced, blood flow was decreased to zero, and the artery was occluded in about 40 to 45 minutes in vehicle-treated animals. Milvexian was associated with a dose-dependent increase in duration of the patency of the carotid artery. FIG. 8 shows the effects of vehicle and milvexian on integrated blood flow in the prevention ECAT rabbit model. Integrated blood flow averaged 11±2% in vehicle-treated animals following thrombus induction. By blocking the formation of thrombus, milvexian was associated with a dose-dependent increase in integrated blood flow. At the highest dose, integrated blood flow was 76±5% of the control level.

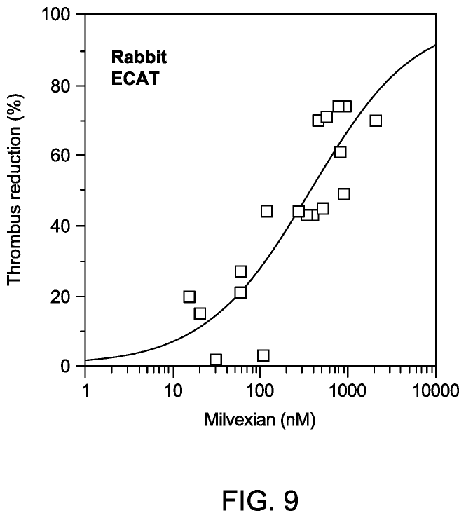

Milvexian was associated with a dose-dependent reduction in thrombus weight. At the highest dose, thrombus weight was reduced by 70±2% of the control level. FIG. 9 shows the concentration-response curve for milvexian in the prevention ECAT model. Milvexian was associated with a concentration-dependent antithrombotic effect with a 20% maximal effective concentration (EC₂₀) of 55 nM (95% confidence interval [CI]=23-128), a EC₅₀ of 375 nM (95% CI=250-561), and a Hill slope of 0.7 (95% CI=0.4-1).

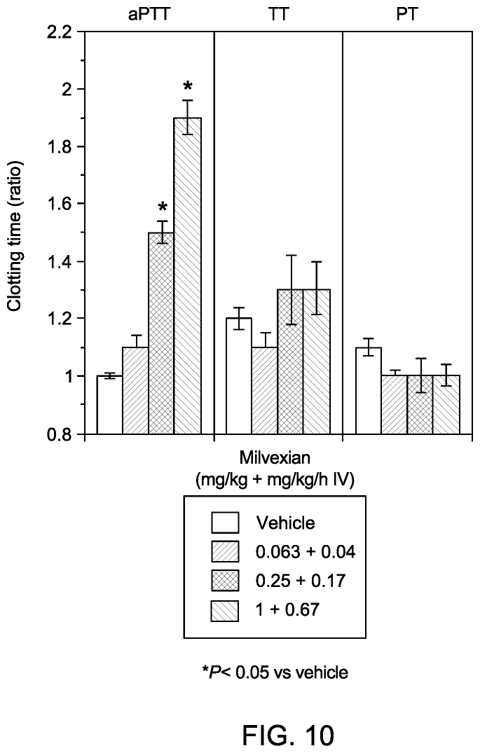

FIG. 10 shows ex vivo effects of milvexian on aPTT, T, and PT.

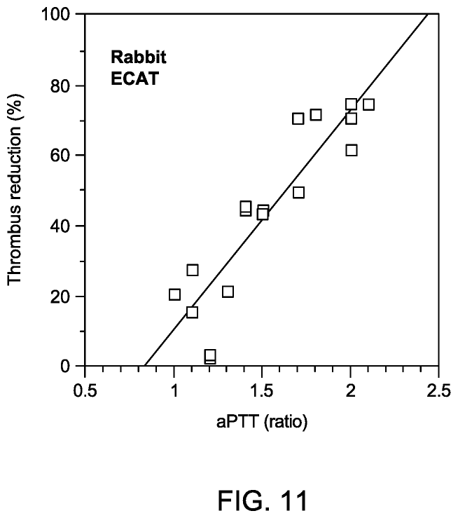

Milvexian elevated aPTT significantly at the highest 2 doses and did not alter TT and PT significantly, consistent with the mechanism of FXIa inhibition. FIG. 11 shows a good correlation (r²=0.83) between the antithrombotic effect of milvexian in the rabbit ECAT and its ex vivo aPTT activity. FIG. 11 also shows that a 1.6-fold prolongation of aPTT may be needed to achieve 50% thrombus weight reduction in this model.

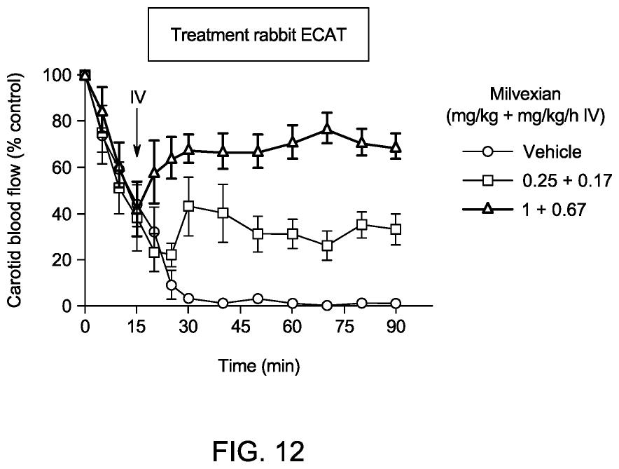

FIG. 12 shows antithrombotic effects of vehicle and milvexian in the treatment ECAT. After the initiation of thrombosis, blood flow was gradually decreased to similar levels at 15 min among different groups. Administration of milvexian at 15 minutes improved the patency of the injured artery in a dose-dependent manner, and carotid blood flow at 90 minutes averaged 1±0.3, 39±10, and 66±2%* in the groups treated with the vehicle and with milvexian 0.25+0.17 and 1+0.67 mg/kg f mg/kg/h, respectively (*P<0.05 vs vehicle; n=6/group, FIG. 12). It also produced a significant dose-dependent reduction in thrombus weight by 25±7% and 61±6% at doses of 0.25+0.17 and 1+0.67 (mg/kg+mg/kg/h, respectively ((*P<0.05; n=6/dose). EC₅₀ of milvexian in the treatment ECAT is 1.06 μM (95% CI=0.76-1.47), which was about 2.8-fold higher than the EC₅₀ obtained in the prevention ECAT (FIG. 9).

In the rabbit ECAT model, milvexian caused a dose-dependent decrease in both clot weight (FIG. 9) and preservation of blood flow without a significant increase in bleeding time. The equivalent to half the maximal effective concentration (EC₅₀) plasma concentrations in the rabbit ECAT model was 235 ng/mL (375 nM). Making corrections for the difference in potency in human versus rabbit FXIa (where milvexian is more potent than rabbit FXIa) and the differences in plasma protein binding between human versus rabbit (where protein binding is lower in human versus rabbit plasma) yields a human target of 34.5 ng/mL (55 nM) as the trough target concentration.

### Example 3

The rabbit ECAT model, described by Wong et al. was used in the rabbit ECAT study (Wang et al. J. Pharmacol. Exp. Ther. 295:212-8, 2002). The ECAT model was conducted 3 to 4 days after the last dose of vehicle, FXI-ASO2, or FXI-ASO1. FXI-ASO1 has the nominal nucleotide sequence of GTAACATGTGCCCTITCCTT (SEQ ID NO: 1) and is complementary to a sequence within rabbit FXI mRNA. FXI-ASO2 (control ASO) has the nominal nucleotide sequence of CCTTCCCTGAAGGTTCCTCC (SEQ ID NO: 2) and has no known complementary sequence in rabbit mRNA. Oligonucleotides were chemically modified with phosphorothioate in the backbone and 2′-O-methoxyethyl on the wings with a central deoxy gap (so-called 5-10-5 design). Cytosine bases were rendered as 5-methylcytosine.

Separate samples for each dose of FXI-ASO1 and FXI-ASO2 were weighed on an analytical balance. The ASO content as a weight percent was taken into account so that the administered dose represented the true amount of FXI-ASO. Immediately prior to dosing, the ASO was dissolved in saline to produce concentrations of 4, 10 or 30 mg/mL to allow subcutaneous administration of doses of 2, 5 or 15 mg/kg in a volume of 0.5 mL/kg. Doses were administered twice weekly for a total of 4 weeks (8 doses) at approximately 3 and one half day intervals.

Male New Zealand White rabbits were anesthetized with ketamine (50 mg/kg+50 mg/kg/h intramuscularly) and xylazine (10 mg/kg+10 mg/kg/h intramuscularly). Thrombosis was induced by electrical stimulation of the carotid artery for 3 min at 4 mA using an external stainless-steel bipolar electrode. Carotid blood flow was measured with an electromagnetic flow probe continuously beginning 30 min before the electrical stimulation and over a 90-min period after stimulation to monitor thrombosis-induced occlusion. Integrated carotid blood flow, as a percentage of pre-electrical stimulation control period, was measured by the area under the flow-time curve. In addition, thrombus from the injured artery was removed, blotted twice on a weighing paper to remove residual fluid, and weighed.

Following electric current stimulation, thrombus formation was induced, blood flow was decreased to zero and the artery was occluded in about 40-45 min in vehicle-treated animals. FXI-ASO1 caused a dose-dependent increase in duration of the patency of the carotid artery.

Relative to the pre-electrical stimulation period, integrated blood flow averaged 12±3% in vehicle-treated animals and 23±8% in FXI-ASO1-treated animals following thrombus induction. By blocking the formation of thrombus, FXI-ASO1 treatment caused a dose-dependent increase in integrated blood flow. At a dose of 2 mg/kg, integrated blood flow was 64±8% of the pre-injury level. Higher doses maintained integrated blood flow at more than 90%.

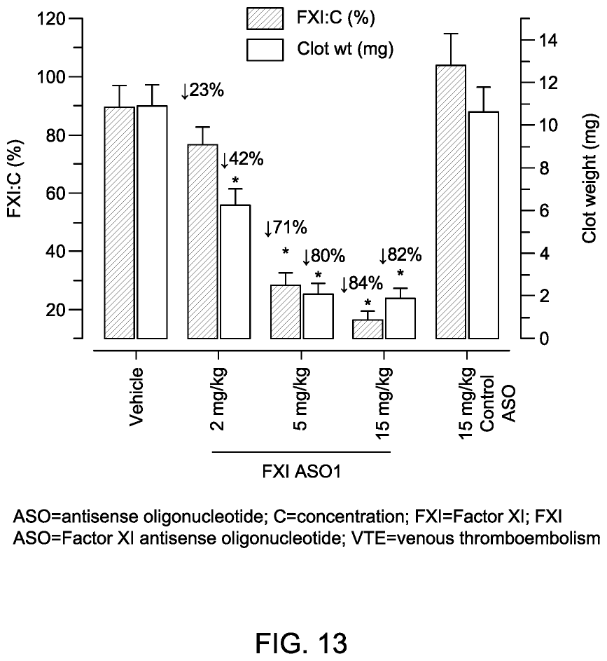

FXI-ASO2 did not reduce thrombus weight. FXI-ASO1 caused a dose-dependent reduction in thrombus weight. At the top dose, thrombus weight was reduced by 82% as compared to vehicle. FIG. 13 shows the dose-dependent decrease in FXI levels and clot weight observed in the rabbit ECAT model with ASO-induced inhibition of FXI.

Reduction of circulating FXI in rabbits exhibited strong antithrombotic efficacy in the prevention of arterial thrombosis in the ECAT model. A 1.3-fold prolongation of aPTT was observed to achieve an 80% thrombus weight reduction in the rabbit ECAT model. Factor XI clotting activity as measured by the coagulation assay was well correlated to plasma FXI concentration as determined by mass spectrometry. In the rabbit ECAT model, a reduction of FXI:C to less than 30% of normal resulted in near complete preservation of integrated blood flow and thrombus weight reduction of 80% or greater.

Based on data obtained from healthy subjects, population PK modeling indicates that doses likely to achieve the trough concentration targets based on apixaban and ASO for a VTE prevention study are between 100 to 200 mg once daily and 25 to 50 mg BID. A 50-mg once daily dose prolongs the aPTT 1.5-fold for about 24 hours in healthy volunteers and the plasma concentrations achieved are above those required for antithrombotic efficacy (EC50) in the rabbit ECAT model of thrombosis for a sustained period time. The data indicating coverage of the target concentrations in the rabbit ECAT model for both apixaban and the FXI inhibitor FXI-ASO1, supports the potential for milvexian to provide similar or greater efficacy in patients.

1. A method of treating or preventing a venous thromboembolic disorder comprising: orally administering to a patient in need thereof a total daily dose of from 25 mg to 400 mg of milvexian, or pharmaceutically acceptable salt thereof.

2. The method according to claim 1, wherein the total daily dose is from 50 mg to 400 mg.

3. The method according to claim 1, wherein the total daily dose is 25 mg.

4. The method according to claim 1, wherein the total daily dose is 50 mg.

5. The method according to claim 1, wherein the total daily dose is 100 mg.

6. The method according to claim 1, wherein the total daily dose is 150 mg.

7. The method according to claim 1, wherein the total daily dose is 200 mg.

8. (canceled)

9. (canceled)

10. (canceled)

11. The method according to claim 1, wherein the venous thromboembolic disorder is proximal and/or distal deep vein thrombosis, nonfatal pulmonary embolism, or death.

12. (canceled)

13. (canceled)

14. (canceled)

15. A method of preventing post-operative venous thromboembolism events in a patient recovering from surgery, said method comprising administering to said patient a daily dose of milvexian, or pharmaceutically acceptable salt thereof, wherein said administration results in the patient experiencing fewer post-operative venous thromboembolism events than would result from subcutaneous administration to the patient of 40 mg per day of enoxaparin.

16. The method according to claim 15, wherein the patient is recovering from abdominal surgery, knee replacement surgery, or hip replacement surgery.

17. The method according to claim 15, wherein the daily dose of milvexian, or pharmaceutically acceptable salt thereof, is 50 mg.

18. The method according to claim 15, wherein the daily dose of milvexian, or pharmaceutically acceptable salt thereof, is 100 mg.

19. The method according to claim 15, wherein the daily dose of milvexian, or pharmaceutically acceptable salt thereof, is 150 mg.

20. The method according to claim 15, wherein the daily dose of milvexian, or pharmaceutically acceptable salt thereof, is 200 mg.

21. (canceled)

22. (canceled)

23. A method of treating or preventing a venous thromboembolic disorder comprising: orally administering a therapeutically effective amount of milvexian, or pharmaceutically acceptable salt thereof once or twice daily to a patient in need thereof, wherein an incidence rate of total venous thromboembolism events in the patient treated with the milvexian, or pharmaceutically acceptable salt thereof is less than 25%.

24. (canceled)

25. (canceled)

26. (canceled)

27. The method of claim 23, wherein the milvexian, or pharmaceutically acceptable salt thereof is administered at a total daily dose ranging from 50 mg to 400 mg.

28. (canceled)

29. The method of claim 23, wherein the milvexian, or pharmaceutically acceptable salt thereof is administered at the total daily dose of 50 mg.

30. The method of claim 23, wherein the milvexian, or pharmaceutically acceptable salt thereof is administered at the total daily dose of 100 mg.

31. The method of claim 23, wherein the milvexian, or pharmaceutically acceptable salt thereof is administered at the total daily dose of 150 mg.

32. The method of claim 23, wherein the milvexian, or pharmaceutically acceptable salt thereof is administered at the total daily dose of 200 mg.

33. The method of claim 23, wherein the milvexian, or pharmaceutically acceptable salt thereof is administered at the total daily dose of 400 mg.

34. The method of claim 23, wherein milvexian is administered twice daily.

35. (canceled)

36. The method of claim 23, wherein the rate of venous thromboembolism occurs in a dose dependent manner without increasing the risk of bleeding compared with enoxaparin.

37-74. (canceled)
